(19) United States
(12) Patent Application Publication
(10) Pub. No.: US 2026/0188139 A1
(43) Pub. Date: Jul. 2, 2026

(54) PROGRAM EXECUTION DEVICE, TRAINING SYSTEM, AND ASSOCIATED PROGRAM EXECUTION METHOD

(71) Applicant: INDUSTRIAL TECHNOLOGY RESEARCH INSTITUTE, Hsinchu (TW)

(72) Inventors: Chia Hung LIN, Taoyuan City (TW); Jian-Ren CHEN, Zhudong Town (TW); Ho-Hsin LEE, Hsinchu City (TW); Chun-Hao HUNG, Taoyuan City (TW)

(73) Assignee: INDUSTRIAL TECHNOLOGY RESEARCH INSTITUTE, Hsinchu (TW)

(21) Appl. No.: 19/003,783

(22) Filed: Dec. 27, 2024

(51) Int. Cl. G09B 19/00; A61B 5/16

(52) U.S. Cl. CPC G09B 19/00; A61B 5/168

(57) ABSTRACT

A program execution device, a training system, and an associated program execution method are provided. The training system includes a sensing device and the program execution device. The program execution device includes: a program execution module, a concentration calculation module, and a program selection module. The program execution module executes an s-th system-selectable program within a (z−1)-th round program-training duration. The sensing device generates a sensing result of a (z−1)-th round corresponding to the (z−1)-th program-training duration. The concentration calculation module calculates a training-duration concentration level of a (z−1)-th round according to the sensing result of the (z−1)-th round. The program selection module selects a t-th system-selectable program to be executed by the program execution module within a z-th program-training duration according to comparison between the training-duration concentration level of the (z−1)-th round and a preset concentration threshold.

### TECHNICAL FIELD

The present invention relates to a program execution device, a training system, and an associated program execution method, and more particularly to a program execution device, a training system, and an associated program execution method, which are adaptively adjusted according to a training process.

### PRIOR ARTS

Patients with Attention Deficit Hyperactivity Disorder (hereinafter, ADHD) usually have symptoms of inattention, hyperactivity, and impulsivity. Due to staffing constraints, doctors or physical therapists have limited time and opportunities to provide non-pharmacological treatments for patients. Thus, developments of digital training systems that can assist ADHD patients in attention training have been launched for this market.

Usually, a training system provides programs (games) so that the patient can operate the training system at home on a long-term and regular basis. However, the training system often provides the same program content for the patient to practice. The unchanged process easily makes the patient bored during the operation of the training system, thereby reducing the patient's motivation and concentration in training.

Furthermore, when the patient uses the training system at home without the company of a professional person, the patient is likely to give up continuing to operate the training system if the patient has low active anticipation or even loses the willingness to operate the training system. Since the therapeutic effect provided by the training system is positively correlated with the patient's continuous operation duration, the overall training effect provided by the training system will be greatly limited if the patient lacks the willingness to operate the training system or even refuses to use the training system.

Therefore, the conventional training system adopting fixed programs to train the patient cannot encourage the patient to perform continued operations without dynamically responding to the patient's fluctuating concentration state. As a result, the efficacy resulting from the patient's operation of the training system is quite limited. Hence, the conventional training system still needs to be improved.

### SUMMARY OF THE INVENTION

The present invention is directed to a program execution device, a training system, and an associated program execution method. The program execution device, the training system, and the associated program execution method dynamically respond to the requirements of doctor's orders and the user's condition during the training to select proper programs, adjust the difficulty of the programs, and increase the variety of the training process.

According to a first aspect of the present invention, a program execution device is provided. The program execution device includes: a program execution module, a concentration calculation module, and a program selection module. The program execution module executes a (z−1)-th round training-program within a (z−1)-th program-training duration, and executes a z-th round training-program within a z-th program-training duration. The (z−1)-th round training-program is an s-th system-selectable program among Y system-selectable programs. The concentration calculation module is electrically connected to the program execution module. The concentration calculation module calculates a training-duration concentration level of a (z−1)-th round according to a sensing result corresponding to the (z−1)-th program-training duration and being generated by a sensing device. The program selection module is electronically connected to the program execution module and the concentration calculation module. The program selection module includes: a heterogeneous program selection module, a composite program selection module, and a selection strategy judgment module. The heterogeneous program selection module and the composite program selection module are electrically connected to the program execution module. The selection strategy judgment module is electrically connected to the concentration calculation module, the heterogeneous program selection module, and the composite program selection module. The selection strategy judgment module uses one of the heterogeneous program selection module and the composite program selection module to select a t-th system-selectable program among the Y system-selectable programs as the z-th round training-program in response to comparison between the training-duration concentration level of the (z−1)-th round and a preset concentration threshold, wherein z is a positive integer greater than 1, s, t, and Y are positive integers, and s and t are smaller than or equal to Y.

According to a second aspect of the present invention, a training system including a sensing device and a program execution device is provided. The sensing device senses within a (z−1)-th program-training duration to generate a sensing result of a (z−1)-th round. The program execution device includes: a program execution module, a concentration calculation module, and a program selection module. The program execution module executes a (z−1)-th round training-program within a (z−1)-th program-training duration, and executes a z-th round training-program within a z-th program-training duration. The (z−1)-th round training-program is an s-th system-selectable program among Y system-selectable programs. The concentration calculation module is electrically connected to the program execution module. The concentration calculation module calculates a training-duration concentration level of a (z−1)-th round according to the sensing result of the (z−1)-th round. The program selection module is electronically connected to the program execution module and the concentration calculation module. The program selection module includes: a heterogeneous program selection module, a composite program selection module, and a selection strategy judgment module. The heterogeneous program selection module and the composite program selection module are electrically connected to the program execution module. The selection strategy judgment module is electrically connected to the concentration calculation module, the heterogeneous program selection module, and the composite program selection module. The selection strategy judgment module uses one of the heterogeneous program selection module and the composite program selection module to select a t-th system-selectable program among the Y system-selectable programs as the z-th round training-program in response to comparison between the training-duration concentration level of the (z−1)-th round and a preset concentration threshold, wherein z is a positive integer greater than 1, s, t, and Y are positive integers, and s and t are smaller than or equal to Y.

According to a third aspect of the present invention, a program execution method applied to a program execution device is provided. The program execution method includes the following steps. First, a (z−1)-th round training-program is executed within a (z−1)-th program-training duration. The (z−1)-th round training-program is an s-th system-selectable program among Y system-selectable programs. Next, a training-duration concentration level of a (z−1)-th round is calculated according to a sensing result of the (z−1)-th round corresponding to the (z−1)-th program-training duration. A t-th system-selectable program is selected among the Y system-selectable programs as a z-th round training-program in response to comparison between the training-duration concentration level of the (z−1)-th round and a preset concentration threshold. Subsequently, the z-th round training-program is executed within the z-th program-training duration, wherein z is a positive integer greater than 1, s, t, and Y are positive integers, and s and t are smaller than or equal to Y.

For a better understanding of the above and other aspects of the present invention, embodiments are specifically described in detail with reference to the accompanying drawings as follows:

### BRIEF DESCRIPTION OF THE DRAWINGS

FIG. 1 is a schematic diagram showing that a user (usr) operates the training system according to the doctor's orders.

FIGS. 2A-2D are schematic diagrams showing display images of a program.

FIGS. 3A-3D are schematic diagrams showing display images of another program.

FIG. 4 is a schematic diagram showing the relevant data of Y=3 system-selectable programs sysPGM[1]˜sysPGM[3] provided by the training system trnSYS.

FIG. 5 is a block diagram illustrating a training system trnSYS.

FIG. 6 is a block diagram illustrating a program similarity calculation module.

FIGS. 7A and 7B are flowcharts showing the program execution method executed by a program execution device, for allowing a user (usr) to perform this day's program training.

FIG. 8 is a flowchart showing that a heterogeneous program selection module selects a z-th round training-program rnd_trnPGM[z]=sysPGM[t] according to the program similarity degrees pgmSIM(1˜Y, s)=pgmSIM(s, 1˜Y) between the system-selectable programs sysPGM[1]˜sysPGM[Y] and the (z−1)-th round training-program rnd_trnPGM[z−1]=sysPGM[s].

FIG. 9 is a block diagram illustrating a composite program selection module.

FIG. 10 is a flowchart showing that the composite program selection module selects the z-th round training-program rnd_trnPGM[z]=sysPGM[t] according to a composite selection rule.

FIG. 11 is a flowchart showing that a level adjustment module selectively adjusts the program execution levels sysPGM_curLVL[sysPGM[1]]˜sysPGM_curLVL[sysPGM[Y]] corresponding to the system-selectable programs sysPGM[1]˜sysPGM[Y].

FIG. 12 is a flowchart showing that a training-volume calculation module calculates the y-th program expected training-volume pgm_expVOL(z, sysPGM[y]) of the z-th round corresponding to the system-selectable program sysPGM[y] when the system-selectable program sysPGM[y] is selected as the z-th round training-program rnd_trnPGM[z]=sysPGM[y] for the z-th program-training duration.

FIG. 13 is a block diagram illustrating a training result calculation module.

FIG. 14 is a flowchart showing how the training result calculation module calculates the day's remaining training-volume rnd_balVOL[z] of the z-th round.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

As described above, the training system of prior arts has the defect of being unable to encourage patients to operate continuously. To resolve such defect, the present disclosure proposes a training system trnSYS for an ADHD patient to undergo digital therapy. The training system trnSYS of the present disclosure can dynamically adjust the training content according to the user's concentration and the completion rate of the programs during the patient's practice period, so as to encourage the patient (the user (usr) of the training system trnSYS) to receive digital therapy continuously.

The training system trnSYS of the present disclosure provides X training aspects trnASP[1]˜trnASP[X] based on X training objectives, wherein x and X are positive integers and 1≤x≤X. For illustration purposes, it is supposed X=5 in the present disclosure. Please refer to Table 1, which gives an example with training aspects trnASP[1]˜trnASP[5] based on X=5 training objectives and provided by the training system trnSYS in an embodiment of the present disclosure.

TABLE 1

| | Training |
|---|---|
| Training aspect trnASP[x], 1 ≤ x ≤ X | objective |
| trnASP[1] | Distraction |
| | ignoring |
| trnASP[2] | Key point |
| | finding |
| trnASP[3] | Differentiating |
| trnASP[4] | Attention |
| | switching |
| trnASP[5] | Strategy |
| | developing |

The training objective of the first training aspect trnASP[1] is distraction ignoring; the training objective of the second training aspect trnASP[2] is key point finding; the training objective of the third training aspect trnASP[3] is differentiating; the training objective of the fourth training aspect trnASP[4] is attention switching; and the training objective of the fifth training aspect trnASP[5] is strategy developing. In actual applications, the number of training aspects provided by the training system trnSYS may be defined according to the needs of the patient in the doctor's treatment. Therefore, the value of X is not limited.

Before a user (usr) is required to use the training system trnSYS of the present application to perform program training, the doctor prescribes doctor's orders according to the symptoms of the user (usr). The training system trnSYS of the present disclosure can quantificationally convert the doctor's order, including the time duration that the doctor requires the user (usr) to operate the training system trnSYS every day and the training proportions corresponding to the X training aspects trnASP[1]˜trnASP[X] needed by the user (usr), into input parameters predefined by the training system trnSYS. Subsequently, when the user (usr) uses the training system to perform program training, the training system trnSYS can provide programs appropriate for the user (usr) based on the requirements regarding the training duration and the training proportions in the doctor's orders.

In order to convert the contents of the doctor's orders into the input parameters predefined by the training system trnSYS, the training system trnSYS of the present disclosure defines the operating duration per day of the training system trnSYS prescribed in the doctor's orders for the user (usr) as the requested daily training duration reqTrnDURpD[usr] of the user (usr); and defines the proportions of the X training aspects trnASP[1]˜trnASP[X] needed by the user (usr) as the user training-aspect contributing ratio usr_allASPr[usr]. These input parameters predefined by the training system trnSYS can be set on the training system trnSYS by a professional person such as a doctor in advance, and then let the user (usr) operate the training system trnSYS. Alternatively, the training system trnSYS may be in signal communication with a network, and a professional person such as a doctor can remotely set or modify the parameters based on the patient's different needs during the disease progression. Furthermore, the training system trnSYS can provide relevant records of the daily training process of the user (usr), so as to help the doctor in observing and analyzing the treatment results. This part is related to variations of the applications, and a detailed description is not given herein.

For illustration purposes, the following training processes are all based on the same user (usr). In actual applications, the training system trnSYS may allow multiple users (usr) (for example, the user usr A, usrB, usrC, and so forth.) to perform training. Consequently, the training system trnSYS should store records of personalized parameters relevant to multiple users (usr).

For example, because patients have respective conditions, the doctor prescribes different requested daily training durations reqTmnDURpD[usr] for the users usrA, usrB, and usrC. For example, the doctor prescribes a requested daily training duration reqTrnDURpD[usrA] of 30 minutes for the user usrA, prescribes a requested daily training duration reqTrnDURpD[usrB] of 20 minutes for the user usrB, and prescribes a requested daily training duration reqTrnDURpD[usrC] of 40 minutes for the user usrC (that is, reqTrnDURpD[usrA]=30, reqTrnDURpD[usrB]=20 and reqTmnDURpD[usrC]=40). This part is related to variations of the applications, and a detailed description is not given in the present disclosure.

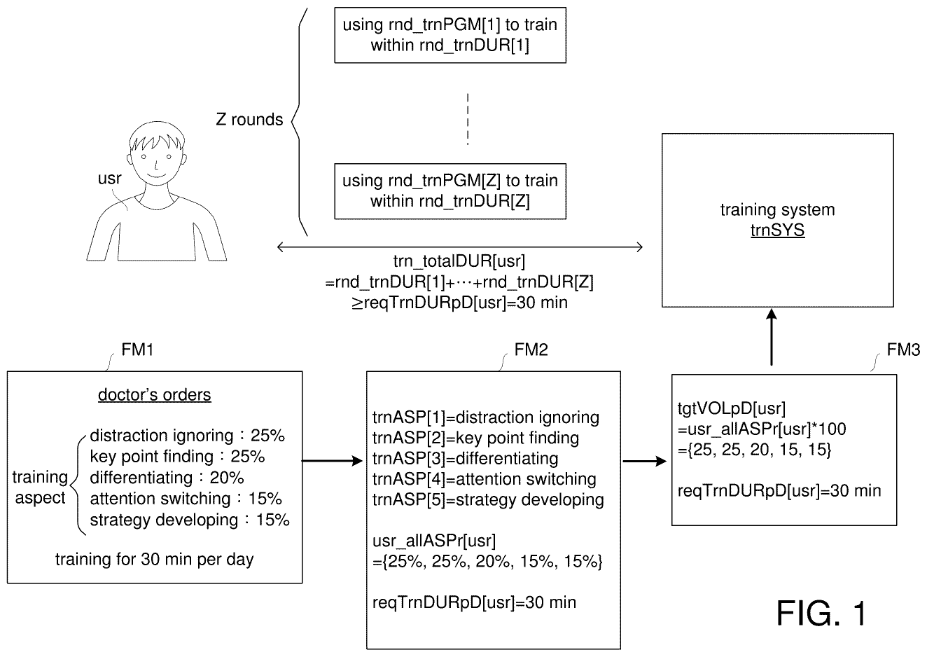

Please refer to FIG. 1, which is a schematic diagram showing that the training system trnSYS converts the doctor's orders for the user (usr) into the input conditions for the training system trnSYS and provides the user (usr) with Z rounds of training. The box FM1 gives the doctor's orders for the user (usr) in this embodiment. The doctor's orders prescribe how the user (usr) operates the training system trnSYS in a single day. Hence, the examples in the following descriptions are based on the fact that the training system provides the training to be performed in a single day.

In the embodiment, it is supposed that the requested daily training duration reqTrnDURpD[usr] of the user (usr) is required to be 30 minutes in the doctor's orders, that is, reqTmnDURpD[usr]=30. The training duration trn_totalDUR[usr] that the user (usr) actually uses the training system trnSYS on that day should be longer than or equal to the requested daily training duration reqTmnDURpD[usr] of the user (usr), that is, trn_totalDUR[usr] ≥reqTmnDURpD[usr]. In the following embodiments, it is supposed in the present disclosure that the training duration trn_totalDUR[usr] that the user (usr) actually uses the training system trnSYS on that day is equal to the requested daily training duration reqTrnDURpD[usr] of the user (usr), that is, trn_totalDUR[usr]=reqTrnDURpD[usr].

Further, in FIG. 1, it is supposed that doctor's orders state that the user (usr) should perform the training regarding the training aspect trnASP[1] to achieve a user training-aspect ratio usr_trnASPr(usr, trnASP[1]) of 25%, that is, usr_trnASPr(usr, trnASP[1])=25%. The user (usr) should perform the training regarding the training aspect trnASP[2] to achieve a user training-aspect ratio usr_trnASPr(usr, trnASP[2]) of 25%, that is, usr_trnASPr(usr, trnASP[2])=25%. The user (usr) should perform the training regarding the training aspect trnASP[3] to achieve a user training-aspect ratio usr_trnASPr(usr, trnASP[3]) of 20%, that is, usr_trnASPr(usr, trnASP[3])=20%. The user (usr) should perform the training regarding the training aspect trnASP[4] to achieve a user training-aspect ratio usr_trnASPr(usr, trnASP[4]) of 15%, that is, usr_trnASPr(usr, trnASP[4])=15%. The user (usr) should perform the training regarding the training aspect trnASP[5] to achieve a user training-aspect ratio usr_trnASPr(usr, trnASP[5]) of 15%, that is, usr_trnASPr(usr, trnASP[5])=15%. Table 2 lists the user training-aspect ratios usr_trnASPr(usr, trnASP[1])˜usr_trnASPr(usr, trnASP[5]) stated in the doctor's orders shown in FIG. 1.

TABLE 2

| Training aspect | Training | User training-aspect ratio |
|---|---|---|
| trnASP[x], 1 ≤ x ≤ X | objective | usr_trnASPr(usr, trnASP[y]) |
| trnASP[1] | Distraction | usr_trnASPr(usr, trnASP[1]) = 25% |
| | ignoring | |
| trnASP[2] | Key point | usr_trnASPr(usr, trnASP[2]) = 25% |
| | finding | |
| trnASP[3] | Differentiating | usr_trnASPr(usr, trnASP[3]) = 20% |
| trnASP[4] | Attention | usr_trnASPr(usr, trnASP[4]) = 15% |
| | switching | |
| trnASP[5] | Strategy | usr_trnASPR(usr, trnASP[5]) = 15% |
| | developing | |

According to concepts of the present disclosure, the set of the user training-aspect ratios usr_trnASPr(usr, trnASP[1])˜usr_trnASPr(usr, trnASP[X]) is defined as the user training-aspect contributing ratio usr_allASPr[usr], that is, usr_allASPr[usr]={usr_trnASPr(usr, trnASP[1]), usr_trnASPr(usr, trnASP[X])}. For example, in FIG. 1, the user training-aspect contributing ratio usr_allASPr[usr]={25%, 25%, 20%, 15%, 15%}. The sum of the user training-aspect ratios usr_trnASPr(usr, trnASP[1])˜usr_trnASPr(usr, trnASP[X]) is 100%, that is, usr_trnASPr(usr, trnASP[1])+ . . . usr_trnASPr(usr, trnASP[X])=100%.

Based on the user training-aspect contributing ratio usr_allASPr[usr]={25%, 25%, 20%, 15%, 15%}, the training system trnSYS can further calculate the target daily training-volume tgtVOLpD[usr] of the user (usr) and express it in a vector format as tgtVOLpD[usr]=usr_allASPr[usr]*100=[usr_trnASPr(usr, trnASP[1])*100, usr_trnASPr(usr, trnASP[X])*100]. For example, in FIG. 1, the target daily training-volume tgtVOLpD[usr]=[25%*100, 25%*100, 20%*100, 15%*100, 15%*100]=tgtVOLpD[usr]=[25, 25, 20, 15, 15].

The training system trnSYS provides Y system-selectable programs sysPGM[1]˜sysPGM[Y] to allow the user (usr) to perform Z rounds of program training. The execution duration of the z-th round of program training is defined as the z-th program-training duration rnd_tmnDUR[z].

For illustration purposes, in the embodiments of the present application, it is supposed that the Z rounds of program-training durations are equal in length. For example, the first to the Z-th program-training durations rnd_trnDUR[1]˜rnd_trnDUR[Z] are all 10 minutes (rnd_trnDUR[1]= . . . =rnd_trnDUR[Z]=10). In actual applications, the program-training durations rnd_trnDUR[z] may be unequal in length.

Further, the present disclosure refers to the program, provided by the training system trnSYS to allow the user (usr) to perform the program training within the z-th program-training duration rnd_trnDUR[z], as the z-th round training-program rnd_trnPGM[z]. The ratio of the z-th program-training duration rnd_trnDUR[z] to the requested daily training duration reqTrnDURpD[usr] of the user (usr) is referred to as the program-training duration-ratio rnd_trnDURr[z] of the z-th round, that is, rnd_trnDURr[z]=rnd_trnDUR[z]/reqTrnDURpD[usr].

Following the supposition that the training duration trn_totalDUR[usr] that the user (usr) actually uses the training system trnSYS on that day is equal to the requested daily training duration reqTrnDURpD[usr] of the user (usr), the sum of the program-training duration-ratios rnd_trnDURr[1]˜rnd_trnDURr[Z] of the first round to the Z-th round is 100% according to the definition of the program-training duration-ratio rnd_trnDURr[z] of the z-th round.

The z-th round training-program rnd_trnPGM[z] could be any one of the system-selectable programs sysPGM[1]˜sysPGM[Y], wherein y, Y, z and Z are positive integers, 1≤z≤Z and 1≤y≤Y. It is to be noted that the variable y represents the identification number/serial number of the system-selectable program sysPGM[y] in the training system trnSYS, and is irrelevant to the selection sequence used by the training system trnSYS.

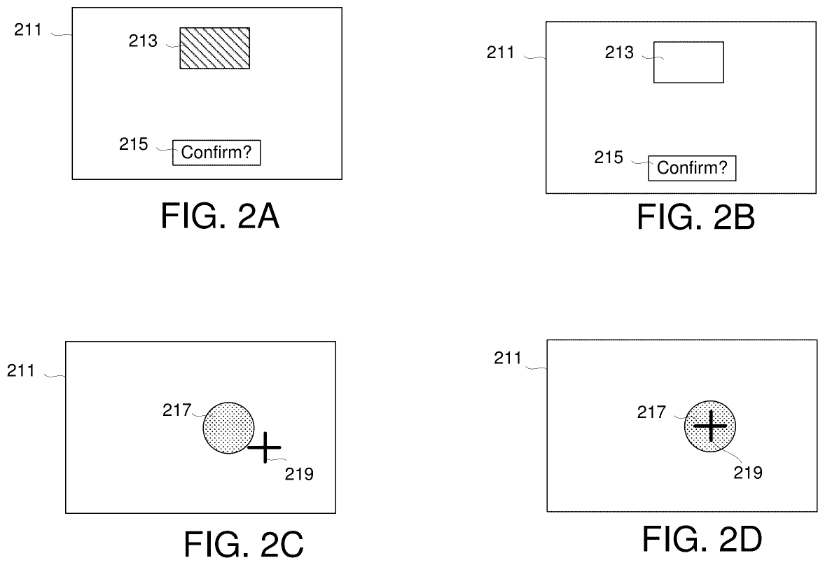

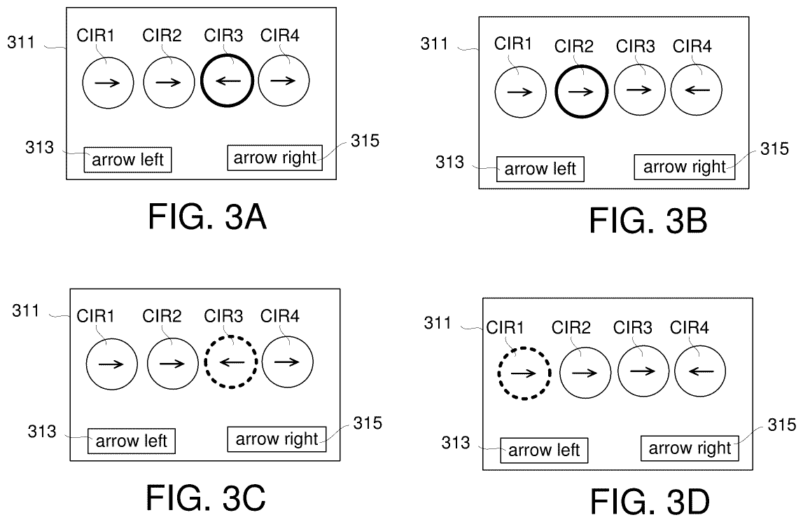

For illustration purposes, it is supposed, in the embodiments of the present disclosure, that the training system trnSYS provides Y=3 system-selectable programs sysPGM[1]˜sysPGM[3]. For example, the system-selectable program sysPGM[1] is Flowing the Circle (see FIGS. 2A-2D), the system-selectable program sysPGM[2] is Flanker Effect (see FIGS. 3A-3D), and the system-selectable program sysPGM[3] is Gomoku. In actual applications, the quantity, program content, and type of the system-selectable programs sysPGM[1]˜sysPGM[Y] need not be limited.

Please refer to FIGS. 2A-2D, which are schematic diagrams showing display images of the system-selectable program sysPGM[1] (Following the Circle). In FIGS. 2A and 2B, the user (usr) needs to observe the color change of the block 213 to determine whether to press the confirmation key 215. When the block 213 has a color other than white, as shown on the screen 211, the user (usr) has to press the confirmation key 215. Otherwise, when the color of the box 213 is white, as shown on the screen 211, the user (usr) should not press the confirmation key 215. The score of the user (usr) using the system-selectable program sysPGM[1] to perform the program training can be determined based on whether the user (usr) appropriately presses or does not press the confirmation key 215.

In FIGS. 2C and 2D, a circle 217 and a cross symbol 219 are displayed on the screen 211. The circle 217 on the screen 211 will automatically change its position dynamically at different speeds and directions, and the user (usr) can control the position of the cross symbol 219 through a joystick. The user (usr) has to move the cross symbol 219 to the center of the circle 217 according to the position of the circle 217. The score of the user (usr) using the system-selectable program sysPGM[1] to perform the program training can be determined based on how much time the user (usr) spends moving the cross symbol 219 to the center of the circle 217.

Please refer to FIGS. 3A-3D, which are schematic diagrams showing display images of the system-selectable program sysPGM[2] (Flanker Effect). In FIGS. 3A-3D, four circles CIR1, CIR2, CIR3, CIR4, and two buttons 313, 315 are displayed on the screen 311. There is one arrow in each circle CIR1, CIR2, CIR3, CIR4 wherein a selected circle (for example, the circle CIR3 in FIG. 3A, the circle CIR2 in FIG. 3B, the circle CIR3 in FIG. 3C and the circle CIR1 in FIG. 3D) has an arrow direction opposite to other unselected circles. At this time, the user (usr) has to press the button 313 or 315 corresponding to the arrow direction of the circle.

In a more advanced manner, the training system trnSYS may require the user (usr) to press one of the buttons 313, 315 based on the frame style of the circle (for example, bold frame, dotted frame). For example, when the selected circle has a bold frame, the user (usr) should select one of the buttons 313, 315 consistent with the arrow direction of the circle. On the contrary, when the selected circle has a dotted frame, the user (usr) has to select one of the buttons 313, 315, indicating the opposite direction to the arrow direction of the circle.

Based on this rule, in FIG. 3A, when the circle CIR3 has a bold frame and the arrow in the circle CIR3 points to the left, the user (usr) should press the button 313; and in FIG. 3B, when the circle CIR2 has a bold frame and the arrow in the circle CIR2 points to the right, the user (usr) should press the button 315. On the other hand, in FIG. 3C, when the circle CIR3 has a dotted frame and the arrow in the circle CIR3 points to the left, the user (usr) should press the button 315; and in FIG. 3D, when the circle CIR1 has a dotted frame and the arrow in the circle CIR1 points to the right, the user (usr) should press the button 313. The system-selectable program sysPGM[2] allows the user (usr) to practice identifying circles with higher importance and needs conversion ability.

The system-selectable programs sysPGM[1]˜sysPGM[3] could be designed by the developers of the training system trnSYS in collaboration with professional persons such as doctors. Professional persons such as doctors can assist in evaluating Y=3 sets of program training-aspect contributing ratios pgm_allASPr[sysPGM[1]]˜pgm_allASPr[sysPGM[3]] regarding X=5 training aspects trnASP[1]˜trnASP[X] that the system-selectable programs sysPGM[1]˜sysPGM[3] can provide. The program training-aspect contributing ratios pgm_allASPr[sysPGM[1]]˜pgm_allASPr[sysPGM[3]] of the system-selectable programs sysPGM[1]˜sysPGM[3] will be served as reference data built in the training system trnSYS.

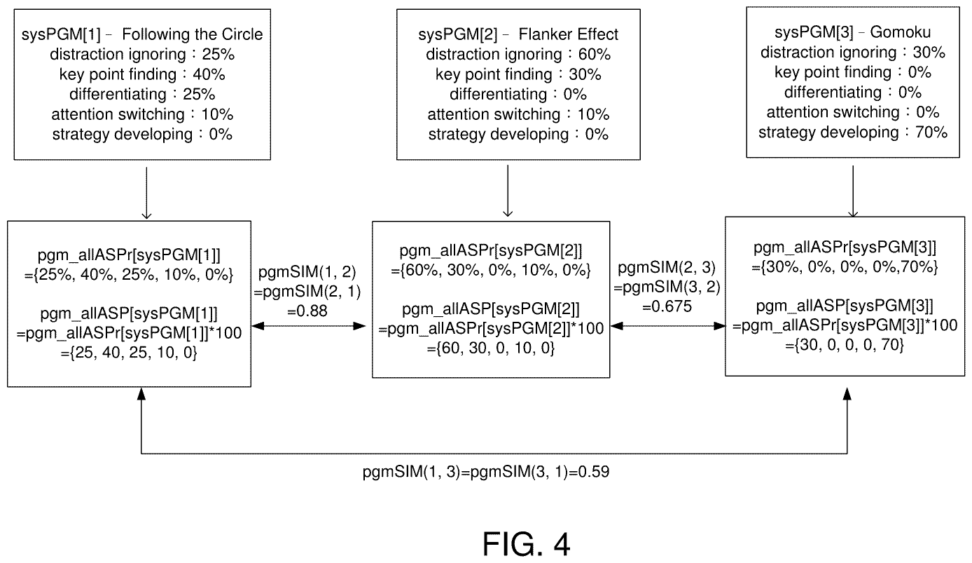

Please refer to FIG. 4, which is a schematic diagram showing the relevant data of Y=3 system-selectable programs sysPGM[1]˜sysPGM[3] provided by the training system trnSYS. Table 3 summarizes, as listed in FIG. 4, the program training-aspect contributing ratios pgm_allASPr[sysPGM[1]]˜pgm_allASPr[sysPGM[3]] regarding X=5 training aspects trnASP[1]˜trnASP[5] provided by the Y=3 system-selectable programs sysPGM[1]˜sysPGM[3] provided by the training system tmnSYS of the present disclosure. After multiplying the X values in the program training-aspect contributing ratios pgm_allASPr[sysPGM[1]]˜pgm_allASPr[sysPGM[3]] by 100, we can get the program training-aspect contributing effects pgm_allASP[sysPGM[1]]˜pgm_allASP[sysPGM[Y]] in a vector format. The program training-aspect contributing effects pgm_allASP[sysPGM[1]]˜pgm_allASP[sysPGM[Y]] in the vector format includes X values corresponding to the X training aspects trnASP[1]˜trnASP[X].

TABLE 3

| | System-selectable | System-selectable | System-selectable |
|---|---|---|---|
| | program sysPGM[1] | program sysPGM[2] | program sysPGM[3] |
| | (Following the Circle) | (Flanker Effect) | (Gomoku) |
| Training aspect | pgmASPr(1, 1) = 25% | pgmASPr(2, 1) = 60% | pgmASPr(3, 1) = 30% |
| trnASP[1] = distraction | | | |
| ignoring | | | |
| Training aspect | pgmASPr(1, 2) = 40% | pgmASPr(2, 2) = 30% | pgmASPr(3, 2) = 0% |
| trnASP[2] = key point | | | |
| finding | | | |
| Training aspect | pgmASPr(1, 3) = 25% | pgmASPr(2, 3) = 0% | pgmASPr(3, 3) = 0% |
| trnASP[3] = differentiating | | | |
| Training aspect | pgmASPr(1, 4) = 10% | pgmASPr(2, 4) = 10% | pgmASPr(3, 4) = 0% |
| trnASP[4] = attention | | | |
| switching | | | |
| Training aspect | pgmASPr(1, 5) = 0% | pgmASPr(2, 5) = 0% | pgmASPr(3, 5) = 70% |
| trnASP[5] = strategy | | | |
| developing | | | |
| Program training-aspect | pgm_allASPr | pgm_allASPr | pgm_allASPr |
| contributing ratio | [sysPGM[1] = | [sysPGM[2] = | [sysPGM[3] = |
| pgm_allASPr[sysPGM[y]] | {25%, 40%, 25%, 10%, | {60%, 30%, 0%, 10%, | {30%, 0%, 0%, |
| | 0%} | 0%} | 0%, 70%} |
| Program training-aspect | pgm_allASP | pgm_allASP | pgm_allASP |
| contributing effect | [sysPGM[1]] = | [sysPGM[2]] = | [sysPGM[3]] = |
| pgm_allASP[sysPGM[y]] | [25, 40, 25, 10, 0] | [60, 30, 0, 10, 0] | [30, 0, 0, 0, 70] |
| Program execution level | sysPGM_curLVL | sysPGM_curLVL[sysPGM[2]] | sysPGM_curLVL[sysPGM[3]] |
| sysPGM_curLVL[sysPGM | [sysPGM[1]] | | |
| [y]] | | | |

According to concepts of the present disclosure, each system-selectable program sysPGM[1]˜sysPGM[Y] corresponds to a program execution level sysPGM_curLVL[sysPGM[1]]˜sysPGM_curLVL[sysPGM[Y]]. The program execution level sysPGM_curLVL[sysPGM[y]] represents the difficulty of the system-selectable program sysPGM[y] executed by the program execution module 113. The program execution levels sysPGM_curLVL[sysPGM[1]]˜sysPGM_curLVL[sysPGM[Y]] of the system-selectable programs sysPGM[1]˜sysPGM[Y] may be identical or different. When the user (usr) uses the training system trnSYS, the training system can adjust trnSYS dynamically the program execution level sysPGM_curLVL[sysPGM[y]] of the system-selectable program sysPGM[y] (1≤y≤Y) to be within a range between the system program lowest level sys_minLVL and the system program highest level sys_maxLVL, that is, sys_minLVL≤sysPGM_curLVL[sysPGM[y]]≤sys_maxLVL.

In actual applications, the quantity and type of the system-selectable programs sysPGM[1]˜sysPGM[Y] provided by the training system trnSYS are not limited. Also, the scoring criteria, game rules, and the like of the system-selectable programs sysPGM[1]˜sysPGM[Y] need not be limited.

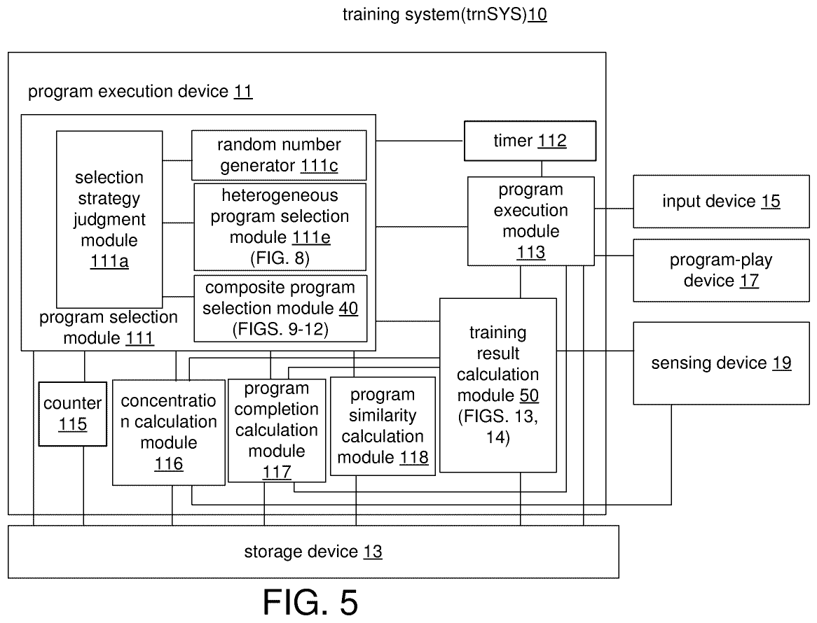

Please refer to FIG. 5, which is a block diagram illustrating the training system trnSYS. The training system (trnSYS) 10 includes: a program execution device 11, a storage device 13, an input device 15, a program-play device 17, and a sensing device 19. The implementation of the storage device 13, the input device 15, the program-play device 17, and the sensing device 19 need not be limited. For example, the input device 15 could be a mouse, a joystick, a touchpad, and so on; the program-play device 17 could be a speaker, a screen, and so on; and the sensing device 19 could be an eye tracker, a wristwatch with an accelerometer, a gravity meter and so on.

In FIG. 5, the program execution device 11 is electrically connected to the storage device 13, the input device 15, the program-play device 17, and the sensing device 19. The program execution device 11 could be implemented by software and/or hardware. In actual applications, the program execution device 11 could be connected to the storage device 13, the input device 15, the program-play device 17, and the sensing device 19 through various types of wired or wireless means. For example, the storage device 13 and the program execution device 11 could be set up on the same computer. Alternatively, the storage device 13 could be a remote server or database in signal communication with the program execution device 11.

The program execution device 11 includes: a program selection module 111, a timer 112, a program execution module 113, a training result calculation module 50, a counter 115, a concentration calculation module 116, a program completion calculation module 117, and a program similarity calculation module 118. The quantities of the counter 115 and the timer 112 are not limited. The program selection module 111 is electrically connected to the storage device 13, the timer 112, the program execution module 113, the training result calculation module 50, the counter 115, the concentration calculation module 116, the program completion calculation module 117, and the program similarity calculation module 118.

In actual applications, the implementation of the program selection module 111, the timer 112, the program execution module 113, the training result calculation module 50, the counter 115, the concentration calculation module 116, the program completion calculation module 117, and the program similarity calculation module 118 is not limited. For example, these components could be implemented by software, hardware, or a combination of both.

The program execution module 113 is configured to execute the training-programs selected by the program selection module 111, and uses the program-play device 17 to output images, audio signals, and the like of the training-programs during the execution of the training-programs. In addition, the program execution module 113 receives the user operation through the input device 15. The training system trnSYS further provides a sensing device 19 around the user (usr) performing the training, and the implementation and the sensing methods of the sensing device 19 are not limited. For example, the sensing device 19 could be configured in a wearable manner, or could be a monitoring device accommodated in the same space as the program execution device 11. Also, the sensing device 19 transmits the sensing results to the program execution device 11 through wired or wireless means.

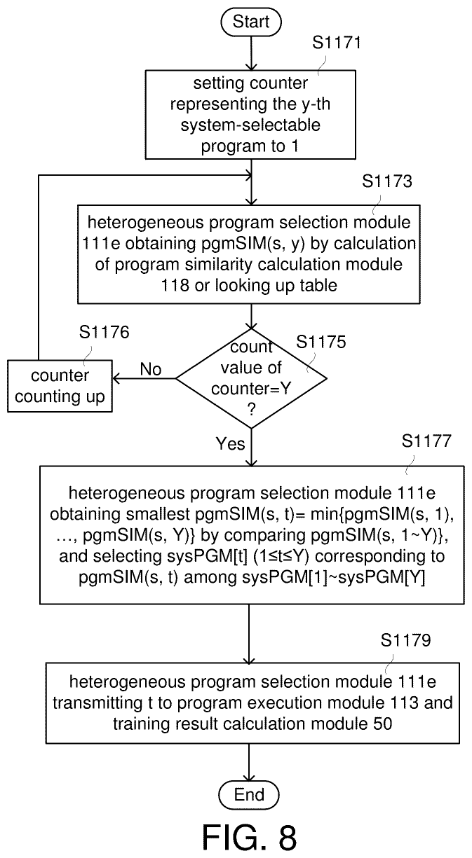

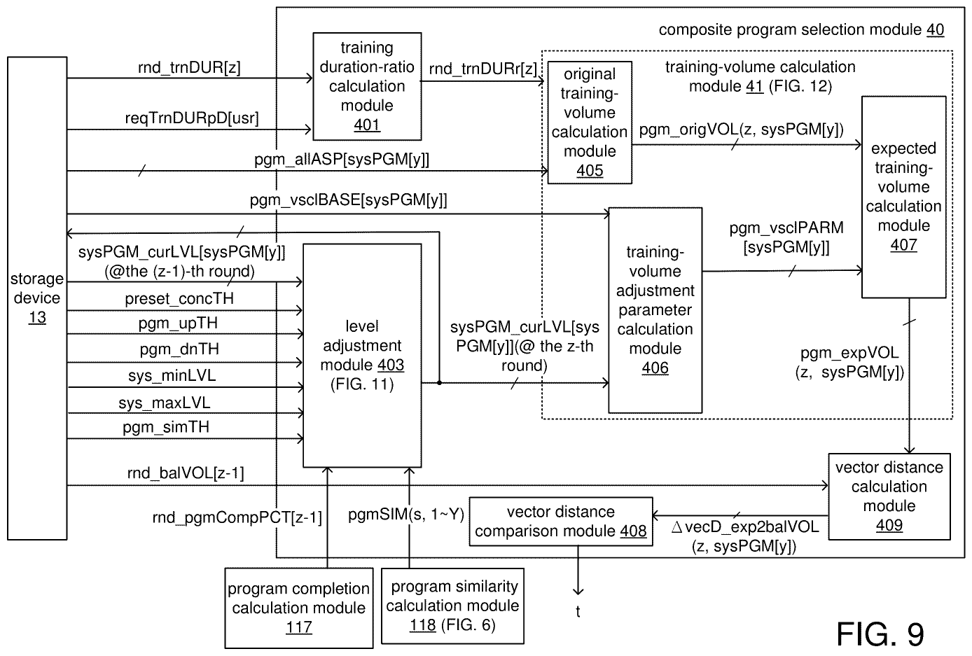

The program selection module 111 further includes: a selection strategy judgment module 111a, a random number generator 111c, a heterogeneous program selection module 111e (FIG. 8), and a composite program selection module 40 (FIG. 9). The random number generator 111c randomly selects value with a range of 1 to Y as a variable t. In the first round (z=1), the selection strategy judgment module 111a transmits the variable t generated by the random number generator 111c to the program execution module 113. In the subsequent round (z>1), the selection strategy judgment module 111a selects one of the heterogeneous program selection module 111e and the composite program selection module 40 to decide the variable t according to the user training condition during the (z−1)-th round of program training. Please further refer to FIG. 8 for the process of selecting the variable t with the heterogeneous program selection module 111e. Please refer to FIGS. 9-12 for the process of selecting the variable t with the architecture of the composite program selection module 40.

The concentration calculation module 116 could be electrically connected to or in signal communication with the sensing device 19. For example, if the sensing device 19 is an eye tracker, the sensing device 19 can sense whether the sight of the user (usr) falls into a specified visual area when the user (usr) performs the program training. The specified visual area corresponds to the display images of the executed programs. The concentration calculation module 116 could be used to calculate the ratio of the number of gaze points located within the specified visual area to the total number of detected gaze points to determine the user concentration.

For example, it is supposed that the training system trnSYS senses K=1000 gaze points within the (z−1)-th program-training duration rnd_trnDUR[z−1]. Among these K=1000 gaze points, J=810 gaze points of the user (usr) are located within the visual area specified by the eye tracker. Thus, the training-duration concentration level rnd_durConcPCT[z−1] of the (z−1)-th round could be expressed as mnd_durConcPCT[z−1]=J/K=810/1000=0.81.

Another example is that the input device 15 is a joystick, and the sensing device 19 is an accelerometer mounted on a wristwatch. At this time, the accelerometer can be used to sense the wrist posture of the user (usr) to determine whether the user (usr) is operating the joystick. In this case, the training-duration concentration level rnd_durConcPCT[z−1] of the (z−1)-th round could be defined as the ratio of the joystick operation time Tjs of the user (usr) within the (z−1)-th round to the (z−1)-th program-training duration rnd_trnDUR[z−1], that is, rnd_durConcPCT[z−1]=Tjs/mnd_trnDUR[z−1].

In actual applications, the method that the concentration calculation module 116 adopts to calculate the user concentration could be adjusted depending on the type of programs and the types of the sensing device 19 and the input device 15 used by the training system trnSYS. For example, the program completion calculation module 117 can calculate the ratio of the scoring result of the program to the perfect score of the program as the program completion rate. In actual applications, each program has its own scoring criteria. Therefore, the program completion calculation module 117 can calculate the program completion rate based on the game rules. This part is related to variations of the applications, and a detailed description is not given herein.

The program execution module 113 is configured to execute the z-th round training-program rnd_trnPGM[z]=sysPGM[t] selected by the program selection module 111. When the program execution module 113 executes the system-selectable program sysPGM[t], the program-play device 17 is configured to display the video of the system-selectable program sysPGM[t] or play the sound of the system-selectable program sysPGM[t]. The input device 15 (for example, mouse, touchpad, joystick, keyboard) allows the user (usr) to control the cursor position, play chess, press the confirmation button, and so forth, for the system-selectable program sysPGM[t]. The sensing device 19 is configured to sense the physical or mental reactions of the user (usr) using the system-selectable program sysPGM[t] within the program-training duration. After the program execution module 113 completes the z-th round of program training, the concentration calculation module 116 and the program completion calculation module 117 calculate the training-duration concentration level rnd_durConcPCT[z] of the z-th round and the training-program completion rate rnd_pgmCompPCT[z] of the z-th round, respectively, according to the sensing results provided by the sensing device 19.

After the concentration calculation module 116 and the program completion calculation module 117 calculate and generate the training-duration concentration level rnd_durConcPCT[z] of the z-th round and the training-program completion rate rnd_pgmCompPCT[z] of the z-th round, the training result calculation module 50 uses these data and sets of the program similarity degrees pgmSIM(1˜Y, 1˜Y) provided by the program similarity calculation module 118 and/or the storage device 13 to analyze the day's remaining training-volume rnd_balVOL[z] after the z-th round. Further, the day's remaining training-volume rnd_balVOL[z] obtained by the training result calculation module 50, the training-duration concentration level rnd_durConcPCT[z] of the z-th round calculated by and obtained from the concentration calculation module 116, and the training-program completion rate mnd_pgmCompPCT[z] of the z-th round calculated by and obtained from the program completion calculation module 117 are provided to the program selection module 111 so that the program selection module 111 can select the (z+1)-th round training-program rnd_trnPGM[z+1] by considering this information.

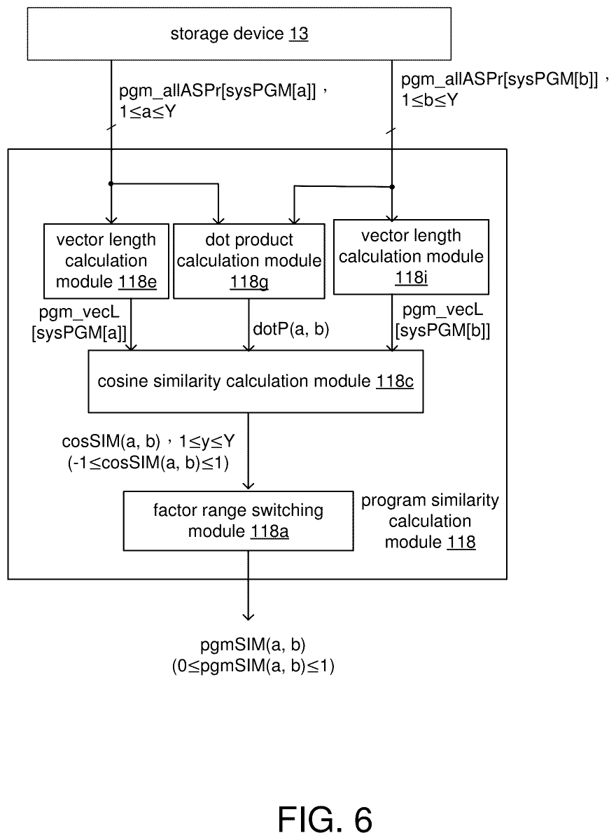

Please refer to FIG. 6, which is a block diagram illustrating a program similarity calculation module. The program similarity calculation module 118 is configured to calculate the program similarity degree pgmSIM(a, b) between any two system-selectable programs sysPGM[a] and sysPGM[b] among the system-selectable programs sysPGM[1]˜sysPGM[Y] of the training system trnSYS, wherein 1≤a≤Y and 1≤b≤Y.

The program similarity calculation module 118 includes: vector length calculation modules 118e, 118i, a dot product calculation module 118g, a cosine similarity calculation module 118c, and a factor range switching module 118a. The vector length calculation modules 118e, 118i are all electrically connected to the dot product calculation module 118g and the cosine similarity calculation module 118c. And, the cosine similarity calculation module 118c is electrically connected to the dot product calculation module 118g and the factor range switching module 118a.

Now, in the above example, the Y=3 sets of program training-aspect contributing ratios pgm_allASPr[sysPGM[1]]={25%, 40%, 25%, 10%, 0%}, pgm_allASPr[sysPGM[2]]={60%, 30%, 0%, 10%, 0%} and pgm_allASPr[sysPGM[3]]={30%, 0%, 0%, 0%, 70%} are expressed in vectors. The program similarity calculation module 118 calculates the program similarity degrees pgmSIM(1˜3, 1˜3) between any two of the system-selectable programs sysPGM[1]˜sysPGM[3] based on these three vectors.

Following the above example and given a=1 and b=3, the program similarity calculation module 118 calculates the program similarity degree pgmSIM(a, b) between the system-selectable programs sysPGM[1] and sysPGM[3] as follows.

First, the vector length calculation module 118e rewrites the program training-aspect contributing ratio pgm_allASPr[sysPGM[1]]={25%, 40%, 25%, 10%, 0%} of the system-selectable program sysPGM[a]=sysPGM[1] into a program training-aspect contributing ratio vector [0.25, 0.4, 0.25, 0.1, 0], and calculates the vector length of the program training-aspect contributing ratio vector [0.25, 0.4, 0.25, 0.1, 0], that is, the vector length thereof pgm_vecL[sysPGM[a]]=(0.25²+0.4²+0.25²+0.1²+0²)^{1/2}=0.5431.

On the other hand, the vector length calculation module 118i rewrites the program training-aspect contributing ratio pgm_allASPr[sysPGM[3]]={30%, 0%, 0%, 0%, 70%} of the system-selectable program sysPGM[b]=sysPGM[3] into a program training-aspect contributing ratio vector [0.3, 0, 0, 0, 0.7], and calculates the vector length of the program training-aspect contributing ratio vector [0.3, 0, 0, 0, 0.7], that is, the vector length thereof pgm_vecL[sysPGM[b]]=(0.3²+0₂+0₂+0₂+0.7₂)^{1/2}=0.7616.

Then, the dot product calculation module 118g calculates the dot product of the vector [0.25, 0.4, 0.25, 0.1, 0] representing the program training-aspect contributing ratio pgm_allASPr[sysPGM[1]]={25%, 40%, 25%, 10%, 0%} of the system-selectable program sysPGM[1] and the vector [0.3, 0, 0, 0, 0.7] representing the program training-aspect contributing ratio pgm_allASPr[sysPGM[3]]={30%, 0%, 0%, 0%, 70%} of the system-selectable program sysPGM[3], thereby obtaining the program dot product dotP(a, b) as seen in Equation (1).

$$dotP ( a , b ) = [ 0.25 , 0.4 , 0.25 , 0.1 , 0 ] \cdot [ 0.3 , 0 , 0 , 0 , 0.7 ] = ( 0.25 * 0.3 ) + 0 + 0 + 0 + 0 = 0.075 \qquad Eq . ( 1 )$$

Next, the cosine similarity calculation module 118c calculates the cosine similarity according to the program dot product dotP(a, b)=0.075 calculated by the dot product calculation module 118g, the vector length of the selectable program pgm_vecL[sysPGM[a]]=0.5431 calculated by the vector length calculation module 118e, and the vector length of the selectable program pgm_vecL[sysPGM[b]]=0.7616 calculated by the vector length calculation module 118i. Equation (2) shows the cosine similarity cosSIM(1, 3) between the system-selectable programs sysPGM[1] and sysPGM[3].

$$\begin{matrix}cos SIM ( a , b ) = cos SIM ( 1 , 3 ) \\ = \frac{d o t P ( a , b )}{pgm\_vecL [ s y s P G M [ a ] ] * pgm\_vecL [ s y s P G M [ b ] ]} \\ = \frac{d o t P ( 1 , 3 )}{pgm\_vecL [ s y s P G M [ 1 ] ] * pgm\_vecL [ s y s P G M [ 3 ] ]} \\ = \frac{0.075}{( 0.5431 * 0.7616 )} = 0.18\end{matrix} \qquad Eq . ( 2 )$$

Then, the factor range switching module 118a switches the cosine similarity cosSIM(a, b) ranging between −1 and 1 into the program similarity degree pgmSIM(a, b) ranging between 0 and 1, as seen in Equation (3).

$$pgmSIM ( a , b ) = \frac{cos SIM ( a , b ) + 1}{2} = \frac{0.18 + 1}{2} = 0.59 \qquad Eq . ( 3 )$$

Similarly, the program similarity calculation module 118 of FIG. 6 can calculate the program similarities based on the program training-aspect contributing ratios pgm_allASPr[sysPGM[1]]˜pgm_allASPr[sysPGM[Y]] corresponding to the system-selectable programs sysPGM[1]˜sysPGM[Y], thereby obtaining the program similarity degrees pgmSIM(a, b) listed in Table 4. The variables a and b range from 1 to Y, respectively. It is supposed Y=3 in Table 4.

TABLE 4

| Program similarity | | | | |
|---|---|---|---|---|
| degree | | | | |
| pgmSIM(a, b) | | System-selectable program | | |
| a = 1~Y, b = 1~Y | | sysPGM[1] | sysPGM[2] | sysPGM[3] |
| System- | sysPGM[1] | pgmSIM(1, | pgmSIM(2, | pgmSIM(3, |
| selectable | | 1) = 1 | 1) = 0.88 | 1) = 0.59 |
| program | sysPGM[2] | pgmSIM(1, | pgmSIM(2, | pgmSIM(3, |
| | | 2) = 0.88 | 2) = 1 | 2) = 0.675 |
| | sysPGM[3] | pgmSIM(1, | pgmSIM(2, | pgmSIM(3, |
| | | 3) = 0.59 | 3) = 0.675 | 3) = 1 |

After calculating the program similarity degrees pgmSIM(a, b) based on the program training-aspect contributing ratios pgm_allASPr[sysPGM[1]]˜pgm_allASPr[sysPGM[3]] of the system-selectable programs sysPGM[1]˜sysPGM[3] listed in Table 4, the program similarity calculation module 118 generates the program similarity degrees pgmSIM(1˜3, 1˜3) as shown in Table 4.

The program similarity degree between the system-selectable programs sysPGM[1] and sysPGM[2] is 0.88 (pgmSIM(1, 2)=pgmSIM(2, 1)=0.88). The program similarity degree between the system-selectable programs sysPGM[2] and sysPGM[3] is 0.675 (pgmSIM(2, 3)=pgmSIM(3, 2)=0.675). The program similarity degree between the system-selectable programs sysPGM[3] and sysPGM[1] is 0.59 (pgmSIM(1, 3)=pgmSIM(3, 1)=0.59).

In actual applications, after the program similarity calculation module 118 calculates the program similarities as listed in Table 4, the calculating results could be stored in the storage device 13 as a lookup table. Alternatively, they are calculated by the program similarity calculation module 118 while the training system trnSYS provides the training to the user (usr). This part is related to variations of the applications, and a detailed description is not given herein.

According to concepts of the present disclosure, the program similarity degrees pgmSIM(1˜Y, 1˜Y) calculated by and obtained from the program similarity calculation module 118 are considered by the heterogeneous program selection module 111e and the composite program selection module 40 for subsequent program selection. Therefore, the program similarity calculation module 118 is electrically connected to both the heterogeneous program selection module 111e and the composite program selection module 40.

For illustration purposes, it is supposed in the embodiments that the z-th round training-program rnd_tmnPGM[z] is the t-th system-selectable program sysPGM[t], and it is supposed that the (z−1)-th round training-program rnd_trnPGM[z−1] is the s-th system-selectable program sysPGM[s]. The variables s and t are positive integers, 1≤s≤Y and 1≤t≤Y. The variables s and t may be identical or different.

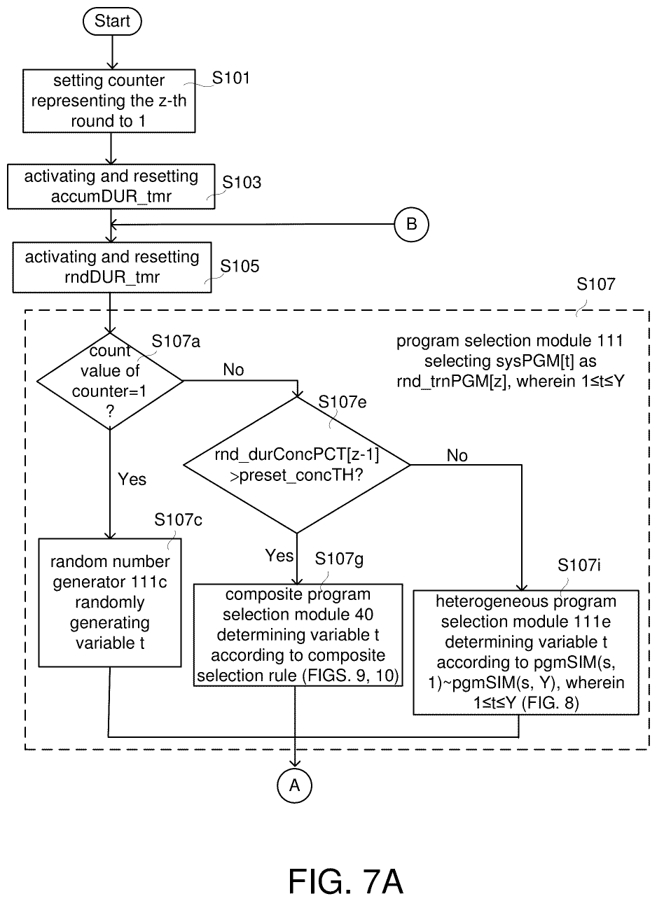

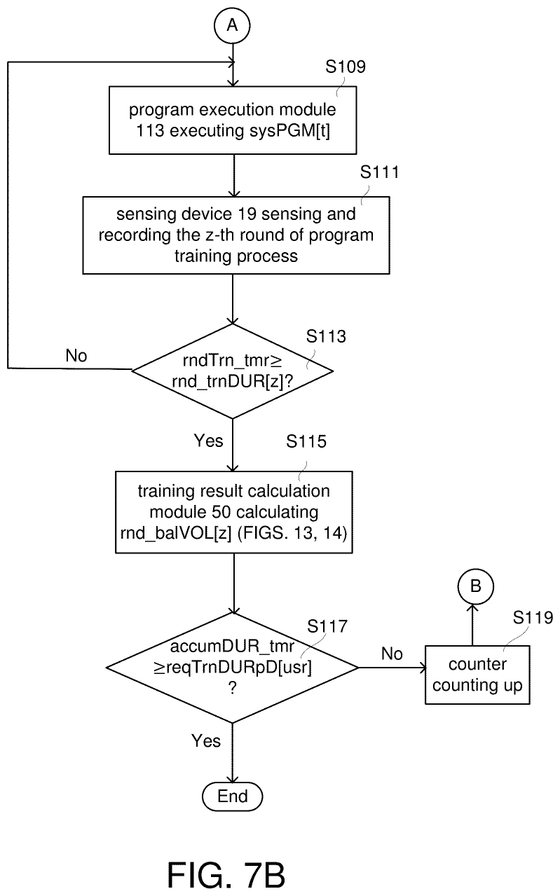

Please refer to FIGS. 7A and 7B, which are flowcharts showing the program execution method performed by a program execution device, for allowing a user (usr) to perform this day's program training. Please also refer to FIGS. 5, 7A and 7B.

First, the selection strategy judgment module 111a initializes the counter 115 representing the z-th round (step S101). Next, the selection strategy judgment module 111a activates and resets the accumulative timer of training duration accumDUR_tmr (step S103) and the single round timer mndDUR_tmr (step S105).

Subsequently, the program selection module 111 selects the system-selectable program sysPGM[t] among the system-selectable programs sysPGM[1]˜sysPGM[Y] as the z-th round training-program rnd_trnPGM[z] (step S107), that is, rnd_trnPGM[z]=sysPGM[t], wherein 1≤t≤Y. The program selection module 111 selects different system-selectable programs sysPGM[t] to meet different conditions. Therefore, step S107 further includes the following steps.

First, the program selection module 111 determines whether the count value of the counter 115 representing the z-th round is equal to 1 (step S107a). If the determination result of step S107a is positive, it means that it is the first round of program training. At this time, the program selection module 111 selects the random number generator 111c to generate the variable t randomly (step S107c). If the determination result of step S107a is negative, the program selection module 111 further determines whether the training-duration concentration level rnd_durConcPCT[z−1] of the (z−1)-th round of the user (usr) within the (z−1)-th program-training duration is greater than a preset concentration threshold preset_concTH (step S107e), that is, determining whether the condition of rnd_durConcPCT[z−1]>preset_concTH is met.

If the determination result of step S107e is negative, the program selection module 111 uses the heterogeneous program selection module 111e to select the system-selectable program sysPGM[t]. In this condition, the heterogeneous program selection module 111e determines the value of the variable t according to the program similarity degrees pgmSIM(s, 1)˜pgmSIM(s, Y) between the system-selectable programs sysPGM[1]˜sysPGM[Y] and the system-selectable program sysPGM[s] (step S107i). Please refer to the description with reference to FIG. 8 for details of step S107i.

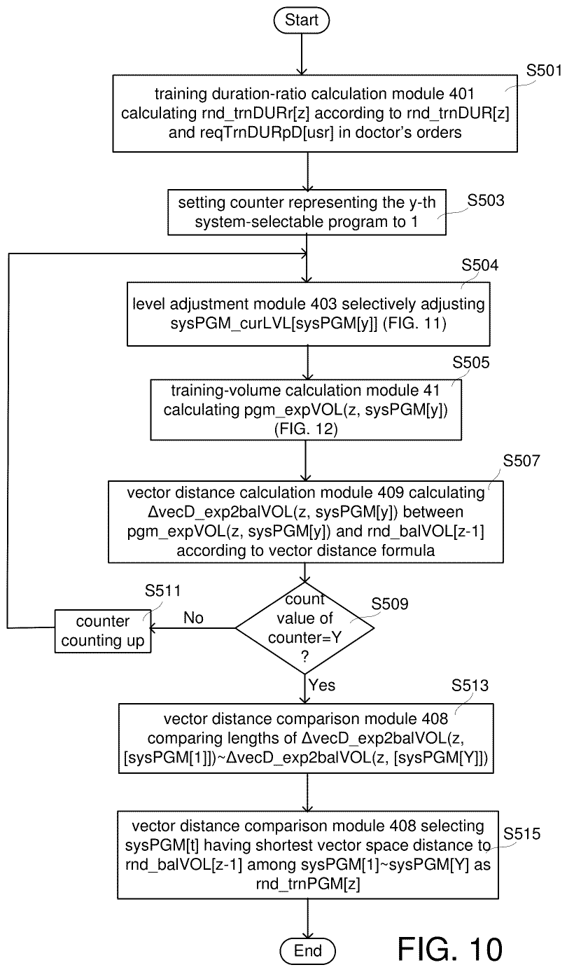

If the determination result of step S107e is positive, the composite program selection module 40 determines the value of the variable t according to a composite selection rule (step S107g). Please further refer to FIGS. 9 and 10 to realize step S107g.

Please refer to Table 5, summarizing the description related to step S107 to show that the selection strategy judgment module 111a selects one of the random number generator 111c, the heterogeneous program selection module 111e, and the composite program selection module 40 as the component for generating the variable t.

TABLE 5

| Determination made by the selection | | |
|---|---|---|
| strategy judgment module 111a to | | |
| select a component for generating | Component for | Related |
| variable t | generating variable t | drawing |
| The first round (z = 1) | Random number | None |
| | generator 111c | |
| The z-th round (z > 1), and the | Heterogeneous | FIG. 8 |
| concentration level of the user(usr) | program selection | |
| performing the [z − 1]-th round training- | module 111e | |
| program rnd_trnPGM[z − 1] = sysPGM[s] | | |
| is insufficient (rnd_durConcPCT[z − | | |
| 1] ≤ preset_concTH) | | |
| The z-th round (z > 1) , and the | Composite program | FIG. 9 |
| concentration level of the user(usr) | selection module 40 | |
| performing the [z − 1]-th round training- | | |
| program rnd_trnPGM[z − 1] = sysPGM[s] | | |
| is sufficient (rnd_durConcPCT[z − | | |
| 1] > preset_concTH) | | |

As described above, the preset concentration threshold preset_concTH is taken in step S107e of the present disclosure to determine whether the user (usr) concentrates on performing training using the system-selectable program sysPGM[s] selected in the (z−1)-th round (sysPGM[s]=rnd_trnPGM[z−1]) within the (z−1)-th program-training duration rnd_trnDUR[z−1]. If it is found that the concentration level of the user (usr) during the (z−1)-th round is insufficient during the (z−1)-th round, the selection strategy judgment module 111a determines that the current state of the user (usr) is not suitable for continuing to use the program similar to the system-selectable program sysPGM[s].

Once the program selection module 111 determines that the training-duration concentration level rnd_durConcPCT[z−1] of the (z−1)-th round of the user (usr) is not good, the program execution device 11 will avoid selecting the system-selectable program similar to the (z−1)-th round training-program rnd_trnPGM[z−1] as the z-th round training-program rnd_trnPGM[z]=sysPGM[t].

FIGS. 4 and 5 are used to explain how step S107 in FIG. 7A is applied to a practical example. Please refer to FIGS. 4, 5, and 7A. As described above, in the first round (z=1), the selection strategy judgment module 111a selects the random number generator 111c to select the first round (z=1) training-program rnd_trnPGM[1].

Following the example with reference to FIG. 4, it is supposed that the training system trnSYS provides system-selectable programs sysPGM[1]˜sysPGM[3]. And, it is supposed that the random number generator 111c randomly selects t=2 in the first round (z=1). Thus, the program execution module 113 executes Flanker Effect (the system-selectable program sysPGM[2]) in the first round (z=1). After the user (usr) completes the first round (z=1) of 10-minute Flanker Effect training, the selection strategy judgment module 111a receives the training-duration concentration level rnd_durConcPCT[1] of the first round (z=1) from the concentration calculation module 116.

For illustration purposes, it is supposed that the preset concentration threshold preset_concTH=0.5. The following gives two conditions of the first-round (z=1) training-duration concentration level rnd_durConcPCT[1]=0.9 and the first-round (z=1) training-duration concentration level rnd_durConcPCT[1]=0.5 to describe how the program selection module 111 selects the second round (z=2) training-program mnd_trnPGM[2] in these two cases.

In the first condition, it is supposed the first-round (z=1) training-duration concentration level rnd_durConcPCT[1]=0.5. Because rnd_durConcPCT[1]=0.5≤preset_concTH=0.5, the program selection module 111 should execute step S107i at this time. Therefore, in this condition, the selection strategy judgment module 111a will use the heterogeneous program selection module 111e to select the second round (z=2) training-program rnd_trnPGM[2].

In the second condition, it is supposed the first-round (z=1) training-duration concentration level rnd_durConcPCT[1]=0.9. Because rnd_durConcPCT[1]=0.9>preset_concTH=0.5, the program selection module 111 should execute step S107g at this time. Therefore, in this condition, the selection strategy judgment module 111a will use the composite program selection module 40 to select the second round (z=2) training-program rnd_trnPGM[2].

According to the above description, the training system trnSYS of the present application selects the second round (z=2) training-program rnd_trnPGM[2] in different ways in response to different training-duration concentration levels rnd_durConcPCT[1] of the first round (z=1) of the user (usr).

After step S107, the program execution module 113 executes the system-selectable program sysPGM[t] within the z-th program-training duration rnd_trnDUR[z] (step S109). While the program execution module 113 executes the system-selectable program sysPGM[t], the sensing device 19 continuously senses and records the physical and mental states of the user (usr) performing the training within the z-th program-training duration rnd_trnDUR[z], thereby generating the sensing results corresponding to the z-th program-training duration md_trnDUR[z] (step S111).

In step S111, the type of the sensing device 19 used by the training system trnSYS needs not to be limited in practical applications. For example, the sensing device 19 may be an eye tracker for sensing the gaze points of the user, or the sensing device 19 may be a gravity meter for sensing the hand gesture of the user (usr). The calculation of the training-duration concentration level rnd_durConcPCT[z] of the z-th round will also vary with the type of the sensing device 19 and the training content of the system-selectable programs sysPGM[1]˜sysPGM[Y]. Therefore, the training system trnSYS of the present disclosure does not need to limit how the concentration calculation module 116 interprets the sensing results obtained from the sensing device 19 and how to calculate the training-duration concentration level rnd_durConcPCT[z] of the z-th round of the user (usr).

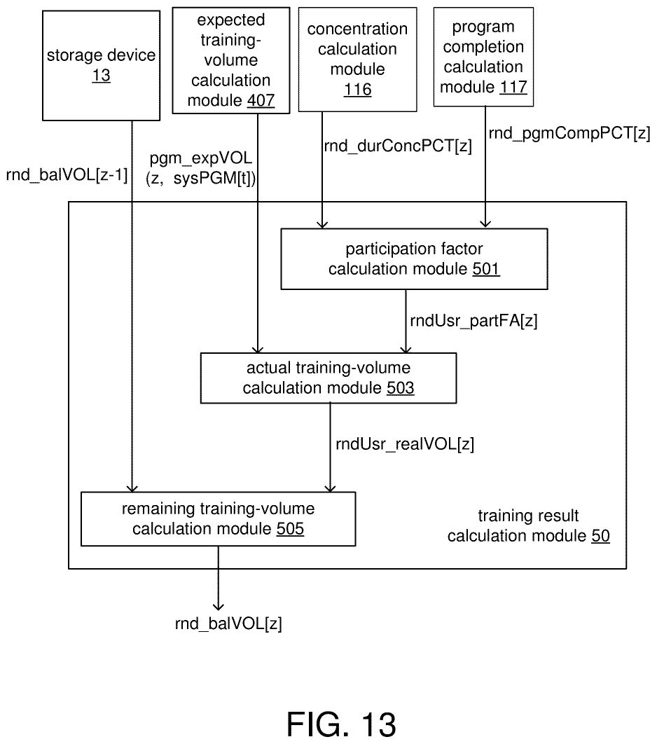

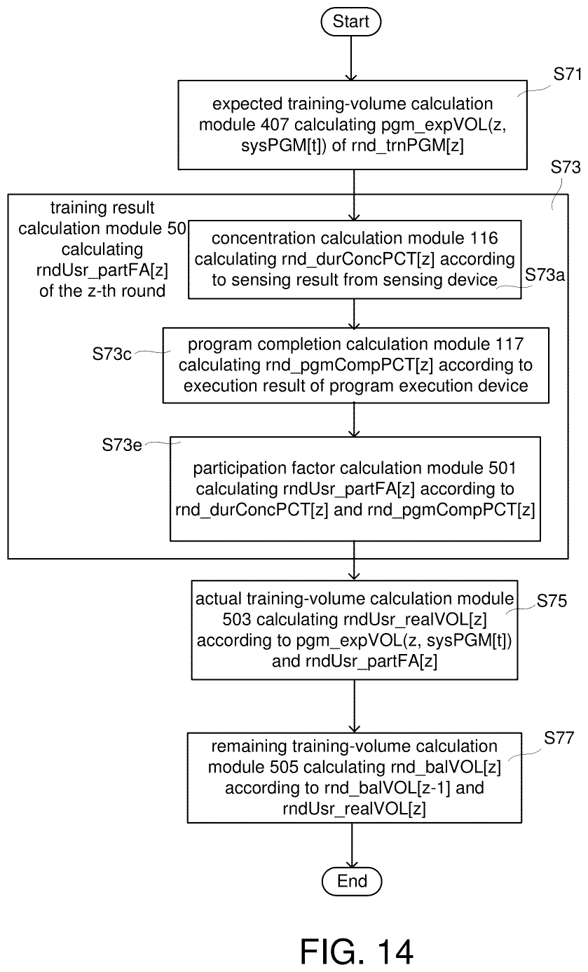

Subsequently, the program selection module 111 determines whether the single round timer mdDUR_tmr is greater than or equal to the z-th program-training duration rnd_trnDUR[z] (step S113), that is, determining whether the condition of rndDUR_tmr>rnd_trnDUR[z] is met. If the determination result of step S113 is negative, the process repeats step S109 and step S111. If the determination result of step S113 is positive, the training result calculation module 50 calculates the day's remaining training-volume mnd_balVOL[z] at the end of the z-th round of program training (step S115). Please refer to the description with reference to FIGS. 13 and 14 for details of step S115.

After completing the z-th round of program training, the program selection module 111 further determines whether the accumulative timer of training duration accumDUR_tmr is greater than or equal to the requested daily training duration reqTrnDURpD[usr] (for example, 30 minutes) of the user (usr) (step S117), that is, determining whether the condition of accumDUR_tmr≥reqTrnDURpD[usr] is met. If the determination result of step S117 is positive, it represents that the requested daily training duration reqTmnDURpD[usr] of the user (usr) has been achieved. Thus, the user (usr) does not need to perform the next round of program training, and the process in FIGS. 7A and 7B ends.

If the determination result of step S117 is negative, it means that the requested daily training duration reqTrnDURpD[usr] of the user (usr) has not been achieved. Therefore, the user (usr) needs to operate the training system trnSYS again to perform the next round of program training. At this time, the program selection module 111 makes the counter 115 representing the z-th round count up (step S119), and then repeats step S107.

It can be seen from the process in FIGS. 7A and 7B that the program execution device 11 divides the day's training process into Z rounds of program training. The program execution device 11 will select the most suitable training-program for the next round of program training at the end of each program training by considering multiple factors, including the requested daily training duration reqTrnDURpD[usr] of the user (usr) in the doctor's orders, the target daily training-volume tgtVOLpD[usr] and the concentration level of the user (usr). Therefore, the training system trnSYS of the present disclosure can flexibly the provide most suitable training-programs rnd_trnPGM[2]˜rnd_trnPGM[Z] for the user (usr) in the subsequent second to Z-th rounds of program training.

Please refer to FIG. 8, which is a flow chart showing that the heterogeneous program selection module selects the z-th round training-program md_trnPGM[z]=sysPGM[t] according to the program similarity degree pgmSIM(1˜Y, s)=pgmSIM(s, 1˜Y) between the system-selectable program sysPGM[1]˜sysPGM[Y] and the (z−1)-th round training-program rnd_trnPGM[z−1]=sysPGM[s]. Please further refer to FIGS. 7A, 7B, and 8. FIG. 8 further illustrates step S107 of FIG. 7A.

First, the heterogeneous program selection module 111e initializes the count value of the counter 115, representing the y-th system-selectable program sysPGM[y] to 1 (step S1171). Next, the heterogeneous program selection module 111e transmits the variables s and y to the program similarity calculation module 118 to calculate the program similarity degree between the system-selectable programs sysPGM[y] and sysPGM[s]. Alternatively, the heterogeneous program selection module 111e can obtain the program similarity degree pgmSIM(y, s)=pgmSIM(s, y) between the system-selectable program sysPGM[y] and the (z−1)-th round training-program rnd_trnPGM[z−1]=sysPGM[s] by looking up the table in the storage device 13 (step S1173).

Subsequently, the heterogeneous program selection module 111e determines whether the count value of the counter 115 representing the y-th system-selectable program sysPGM[y] is equal to Y (step S1175). If the determination result of step S1175 is negative, the counter 115 representing the y-th system-selectable program sysPGM[y] counts up (step S1176), and then the process repeats step S1173. If the determination result of step S1175 is positive, it means that the heterogeneous program selection module 111e has obtained the program similarity degrees pgmSIM(s, 1)˜pgmSIM(s, Y) between the system-selectable programs sysPGM[1]˜sysPGM[Y] and the (z−1)-th round training-program rnd_trnPGM[z−1]=sysPGM[s].

Afterward, the heterogeneous program selection module 111e can sort the program similarity degrees pgmSIM(s, 1)˜pgmSIM(s, Y) between the system-selectable programs sysPGM[1]˜sysPGM[Y] and the (z−1)-th round training-program rnd_trnPGM[z−1]=sysPGM[s] in numerical order. The smallest program similarity degree in the sorting results is represented as pgmSIM(s, t), that is, pgmSIM(s, t)=min {pgmSIM(s, 1), . . . , pgmSIM(s, Y)}. Further, the heterogeneous program selection module 111e selects the system-selectable program sysPGM[t] corresponding to the smallest program similarity degree pgmSIM(s, t) among the system-selectable programs sysPGM[1]˜sysPGM[Y] (step S1177). Afterward, the heterogeneous program selection module 111e transmits the variable t to the program execution module 113 and the training result calculation module 50 (step S1179).

Please refer to FIGS. 7A, 7B, and 8. As described with reference to step S107 of FIG. 7A, when the selection strategy judgment module 111a selects the heterogeneous program selection module 111e to select the z-th round program, it means that the training-duration concentration level rnd_durConcPCT[z−1] of the (z−1)-th round of the user (usr) is lower. In other words, the (z−1)-th round training-program rnd_trnPGM[z]=sysPGM[s] adopted by the training system trnSYS during the (z−1)-th round does not meet the current physical and mental states of the user (usr), so that it cannot encourage the user (usr) to maintain high concentration. In the case that the user (usr) cannot maintain concentration on the (z−1)-th round of program training, the selection strategy judgment module 111a uses the heterogeneous program selection module 111e to select the z-th round training-program rnd_trnPGM[z].

As described with reference to FIG. 8, the heterogeneous program selection module 111e selects the system-selectable program sysPGM[t] that has the smallest program similarity degree relative to the system-selectable program sysPGM[s]. Therefore, if the heterogeneous program selection module 111e selects the z-th round training-program rnd_trnPGM[z]=sysPGM[t] according to the smallest program similarity degree pgmSIM(s, t), the system-selectable program sysPGM[t] selected by the heterogeneous program selection module 111e as the z-th round training-program rnd_trnPGM[z] is surely different from the (z−1)-th round training-program rnd_trnPGM[z−1]=sysPGM[s], that is, s≠t.

Incidentally, in actual applications, after the heterogeneous program selection module 111e sorts the program similarity degrees pgmSIM(s, 1)˜pgmSIM(s, Y) between the system-selectable programs sysPGM[1]˜sysPGM[Y] and the (z−1)-th round training-program rnd_trnPGM[z−1]=sysPGM[s], if there is more than one system-selectable program having the same smallest program similarity degree relative to the system-selectable program sysPGM[s], the heterogeneous program selection module 111e can randomly select any one of these system-selectable programs corresponding to the smallest program similarity degree as the z-th round training-program rnd_trnPGM[z]=sysPGM[t] of the training system trnSYS. This part is related to variations of the applications, and a detailed description is not given in the present disclosure.

Please refer to FIG. 4, Table 4, and step S107 in FIG. 7A and FIG. 8. In short, based on the relationship between the program similarity degrees between the system-selectable programs sysPGM[1]˜sysPGM[3] as shown in FIG. 4 and Table 4, it is supposed that the system-selectable program sysPGM[2] is taken as the first round (z=1) training-program rnd_trnPGM[1]=sysPGM[2] in step S107 of FIG. 7A. After the first round (z=1) of program training ends, two conditions may occur based on the training-duration concentration level rnd_durConcPCT[1] of the first round (z=1) of the user (usr) as follows.

The first condition is that the training-duration concentration level rnd_durConcPCT[1] (for example, 0.5) of the first round (z=1) of the user (usr) is lower than or equal to the preset concentration threshold preset_concTH (for example, 0.5). At this time, the selection strategy judgment module 111a uses the heterogeneous program selection module 111e to select the second round (z=2) training-program rnd_trnPGM[2] (see step S107i of FIG. 7A). It can be seen from FIG. 4 and Table 4 that the program similarity degree pgmSIM(2, 1)=pgmSIM(1, 2) between the system-selectable programs sysPGM[2] and sysPGM[1] is 0.88, that is, pgmSIM(1, 2)=pgmSIM(2, 1)=0.88. And, the program similarity degree pgmSIM(2, 3)=pgmSIM(3, 2) between the system-selectable programs sysPGM[2] and sysPGM[3] is 0.675, that is, pgmSIM(2, 3)=pgmSIM(3, 2)=0.675.

As shown in FIG. 4 and Table 4, the program similarity degree pgmSIM(2, 3)=pgmSIM(3, 2) between the system-selectable programs sysPGM[2] and sysPGM[3] is 0.675, which is lower than 0.88, the program similarity degree pgmSIM(1, 2)=pgmSIM(2, 1) between the system-selectable programs sysPGM[2] and sysPGM[1], that is, pgmSIM(2, 3)=pgmSIM(3, 2)=0.675<(pgmSIM(1, 2)=pgmSIM(2, 1)=0.88. Based on step S1177 of FIG. 8, the heterogeneous program selection module 111e should select the system-selectable program sysPGM[3], corresponding to the lower program similarity degree pgmSIM(2, 3)=pgmSIM(3, 2)=0.675 with the system-selectable program sysPGM[2], I (z=2) training-program rnd_trnPGM[2], that is, rnd_trPGM[2]=sysPGM[3].

The second condition is that the training-duration concentration level rnd_durConcPCT[1] (for example, 0.9) of the first round (z=1) of the user (usr) is higher than the preset concentration threshold preset_concTH (for example, 0.5). At this time, the selection strategy judgment module 111a uses the composite program selection module 40 instead to select the second round (z=2) training-program rnd_trnPGM[2]. Please refer to the description with reference to FIGS. 9-12 to see how the composite program selection module 40 selects the training-program.

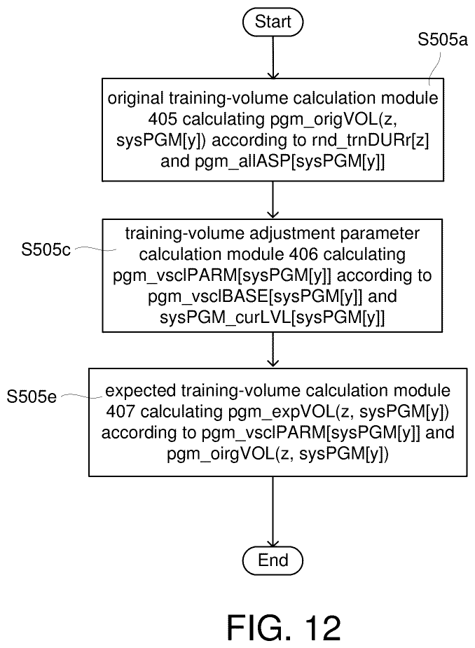

Please refer to FIG. 9, which is a block diagram illustrating the composite program selection module. The composite program selection module 40 includes: a training duration-ratio calculation module 401, a training-volume calculation module 41, a level adjustment module 403, a vector distance calculation module 409, and a vector distance comparison module 408. The training-volume calculation module 41 further includes: an original training-volume calculation module 405, a training-volume adjustment parameter calculation module 406, and an expected training-volume calculation module 407. Please refer to FIG. 12 for the description of the training-volume calculation module 41.

The training duration-ratio calculation module 401 calculates the program-training duration-ratio rnd_trnDURr[z] of the z-th round after receiving the z-th program-training duration rnd_trnDUR[z] and the requested daily training duration reqTrnDURpD[usr] of the user (usr) from the storage device 13, for example, rnd_trnDURr[z]=rnd_trnDUR[z]/reqTrnDURpD[usr].

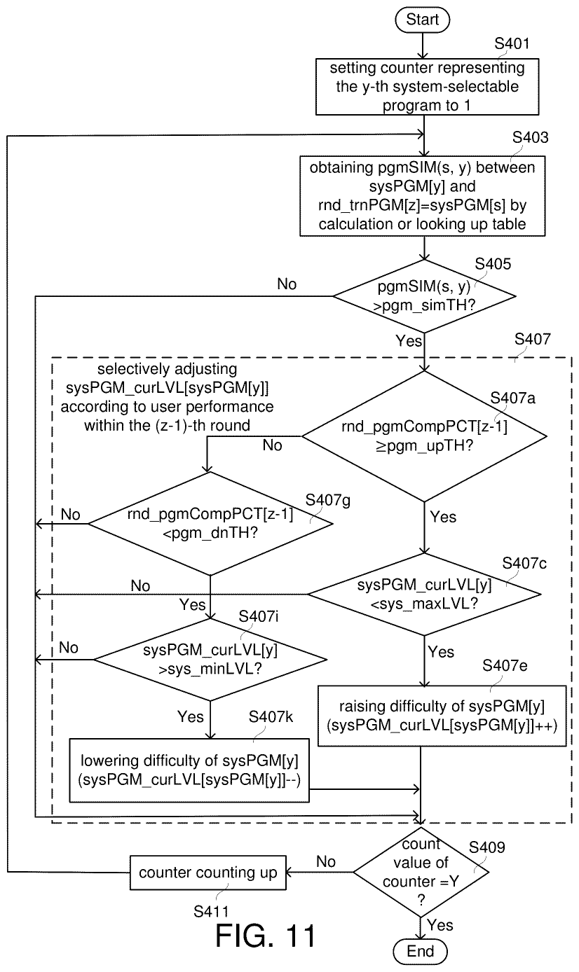

The level adjustment module 403 receives, from the storage device 13, the program execution levels sysPGM_curLVL[sysPGM[1]]˜sysPGM_curLVL[sysPGM[Y]] of the system-selectable programs sysPGM[1]˜sysPGM[Y], the preset concentration threshold preset_concTH, the upgrade threshold of program completion pgm_upTH, the downgrade threshold of program completion pgm_dnTH, the system program lowest level sys_minLVL, the system program highest level sys_maxLVL and the program similarity threshold pgm_simTH set in the (z−1)-th round. Further, the level adjustment module 403 receives the training-program completion rate rnd_pgmCompPCT[z−1] of the (z−1)-th round of the user (usr) from the program completion calculation module 117, and receives the program similarity degrees pgmSIM(s, 1˜Y) corresponding to the system-selectable programs sysPGM[1]˜sysPGM[Y] from the program similarity calculation module 118. The level adjustment module 403 sets the program execution levels sysPGM_curLVL[sysPGM[1]]˜sysPGM_curLVL[sysPGM[Y]] corresponding to the system-selectable programs sysPGM[1]˜sysPGM[Y] again in the z-th round according to the program execution levels sysPGM_curLVL[sysPGM[1]]˜sysPGM_curLVL[sysPGM[Y]] of the system-selectable programs sysPGM[1]˜sysPGM[Y], the preset concentration threshold preset_concTH, the upgrade threshold of program completion pgm_upTH, the downgrade threshold of program completion pgm_dnTH, the system program lowest level sys_minLVL, the system program highest level sys_maxLVL, the program similarity threshold pgm_simTH, the day's remaining training-volume rnd_balVOL[z−1] of the (z−1)-th round, the training-program completion rate rnd_pgmCompPCT[z−1] of the (z−1)-th round of the user (usr) and the program similarity degrees pgmSIM(s, 1˜Y) corresponding to the system-selectable programs sysPGM[1]˜sysPGM[Y]. FIG. 11 further illustrates how the level adjustment module 403 sets the program execution levels sysPGM_curLVL[sysPGM[1]]˜sysPGM_curLVL[sysPGM[Y]] corresponding to the system-selectable programs sysPGM[1]˜sysPGM[Y] in the z-th round.

Next, the components in the training-volume calculation module 41 are described. The original training-volume calculation module 405 calculates the program original training-volumes pgm_origVOL(z, sysPGM[1])˜pgm_origVOL(z, sysPGM[Y]) corresponding to the system-selectable programs sysPGM[1]˜sysPGM[Y] after receiving the program-training duration-ratio rnd_trnDURr[z] of the z-th round from the training duration-ratio calculation module 401, and receiving the program training-aspect contributing effects pgm_allASP[sysPGM[1]]˜pgm_allASP[sysPGM[Y]] corresponding to the system-selectable programs sysPGM[1]˜sysPGM[Y] from the storage device 13. Further, the original training-volume calculation module 405 further transmits the program original training-volumes pgm_origVOL(z, sysPGM[1])˜pgm_origVOL(z, sysPGM[Y]) corresponding to the system-selectable programs sysPGM[1]˜sysPGM[Y] to the expected training-volume calculation module 407 after the calculation is complete.

On the other hand, the training-volume adjustment parameter calculation module 406 receives the program training-volume adjustment bases pgm_vsclBASE[sysPGM[1]]˜pgm_vsclBASE[sysPGM[Y]] corresponding to the system-selectable programs sysPGM[1]˜sysPGM[Y] from the storage device 13, and receives program execution levels the sysPGM_curLVL[sysPGM[1]]˜sysPGM_curLVL[sysPGM[Y]] corresponding to the system-selectable programs sysPGM[1]˜sysPGM[Y] and set by the level adjustment module 403 in the z-th round from the level adjustment module 403. The training-volume adjustment parameter calculation module 406 calculates the program training-volume adjustment parameters pgm_vsclPARM[sysPGM[1]]˜pgm_vsclPARM[sysPGM[Y]] correspond to the system-selectable programs sysPGM[1]˜sysPGM[Y] according to the program training-volume adjustment bases pgm_vsclBASE[sysPGM[1]]˜pgm_vsclBASE[sysPGM[Y]] corresponding to the system-selectable programs sysPGM[1]˜sysPGM[Y] and the program execution levels sysPGM_curLVL[sysPGM[1]]˜sysPGM_curLVL[sysPGM[Y]] of the system-selectable programs sysPGM[1]˜sysPGM[Y], and then transmits the program training-volume adjustment parameters pgm_vsclPARM[sysPGM[1]]˜pgm_vsclPARM[sysPGM[Y]] corresponding to the system-selectable programs sysPGM[1]˜sysPGM[Y] to the expected training-volume calculation module 407.

According to concepts of the present disclosure, the training-volume adjustment parameter calculation module 406 uses the program training-volume adjustment base pgm_vsclBASE[sysPGM[y]] and the program execution level sysPGM_curLVL[sysPGM[y]] of the system-selectable program sysPGM[y] to perform exponentiation operation. The result of the exponentiation operation is taken as the program training-volume adjustment parameter pgm_vsclPARM[sysPGM[y]] corresponding to the system-selectable program sysPGM[y], wherein y=1˜Y.

In the embodiments of the present disclosure, it is supposed that the training-volume adjustment parameter calculation module 406 calculates the program training-volume adjustment parameter pgm_vsclPARM[sysPGM[y]] corresponding to the system-selectable program sysPGM[y] by using the program training-volume adjustment base pgm_vsclBASE[sysPGM[y]] as the base of the exponentiation operation and using the program execution level sysPGM_curLVL[sysPGM[y]] as the exponent of the exponentiation operation, that is, pgm_vsclPARM[sysPGM[y]]=pgm_vsclBASE[sysPGM[y]]^{sysPGM_curLVL[sysPGM[y]]}.

Subsequently, the expected training-volume calculation module 407 calculates the Y number of the program expected training-volumes pgm_expVOL(z, sysPGM[1])˜pgm_expVOL(z, sysPGM[Y]) of the z-th round corresponding to the system-selectable programs sysPGM[1]˜sysPGM[Y] based on the program original training-volumes pgm_origVOL(z, sysPGM[1])˜pgm_origVOL(z, sysPGM[Y]) and the program training-volume adjustment parameters pgm_vsclPARM[sysPGM[1]]˜pgm_vsclPARM[sysPGM[Y]] corresponding to the system-selectable programs sysPGM[1]˜sysPGM[Y]. Then, the expected training-volume calculation module 407 transmits the Y program expected training-volumes pgm_expVOL(z, sysPGM[1])˜pgm_expVOL(z, sysPGM[Y]) of the z-th round corresponding to the system-selectable programs sysPGM[1]˜sysPGM[Y] to the vector distance calculation module 409. FIG. 12 further illustrates the process associated with the training-volume calculation module 41.

According to concepts of the present disclosure, the day's remaining training-volume rnd_balVOL[z−1] of the (z−1)-th round and the Y program expected training-volumes pgm_expVOL(z, sysPGM[1])˜pgm_expVOL(z, sysPGM[Y]) of the z-th round are all in a vector format. The vector distance calculation module 409 receives the day's remaining training-volume rnd_balVOL[z−1] of the (z−1)-th round from the storage device 13. Further, the vector distance calculation module 409 calculates the vector space distances between the day's remaining training-volume rnd_balVOL[z−1] of the (z−1)-th round and the Y program expected training-volumes pgm_expVOL(z, sysPGM[1])˜pgm_expVOL(z, sysPGM[Y]) of the z-th round corresponding to the system-selectable programs sysPGM[1]˜sysPGM[Y]. For example, if the system-selectable program sysPGM[y] is selected as the z-th round training-program rnd_trnPGM[z]=sysPGM[y], the vector distance calculation module 409 calculates the vector space distance Δ vecD_exp2balVOL(z, sysPGM[y]) between the y-th program expected training-volume pgm_expVOL(z, sysPGM[y]) of the z-th round in the vector format and the day's remaining training-volume rnd_balVOL[z−1] of the (z−1)-th round in the vector format. In other words, the vector distance calculation module 409 calculates Y vector space distances Δ vecD_exp2balVOL(z, sysPGM[1])˜Δ vecD_exp2balVOL(z, sysPGM[Y]) according to the distance formula of space vectors. The distance formula of space vectors is not described in detail in the present disclosure.

The vector distance calculation module 409 transmits the vector space distances Δ vecD_exp2balVOL(z, sysPGM[1])˜Δ vecD_exp2balVOL(z, sysPGM[Y]) corresponding to the system-selectable programs sysPGM[1]˜sysPGM[Y] to the vector distance comparison module 408. Afterward, the vector distance comparison module 408 selects the system-selectable program sysPGM[t] with the shortest vector space distance Δ vecD_exp2balVOL(z, sysPGM[t]) as the z-th round training-program rnd_trnPGM[z]=sysPGM[t] according to the comparison results of the vector space distances Δ vecD_exp2balVOL(z, sysPGM[1])˜ Δ vecD_exp2balVOL(z, sysPGM[Y]) corresponding to the system-selectable programs sysPGM[1]˜sysPGM[Y]. The detailed description about how the vector distance comparison module 408 compares the vector space distances Δ vecD_exp2balVOL(z, sysPGM[1])˜ Δ vecD_exp2balVOL(z, sysPGM[Y]) is not given in the present disclosure.

It can be seen from FIG. 9 that the composite program selection module 40 selects the system-selectable program sysPGM[t] as the z-th round training-program rnd_trnPGM[z] based on multiple parameters and multiple steps. In other words, the composite program selection module 40 determines the value of the variable t based on the composite selection rule. The z-th round training-program rnd_trnPGM[z]=sysPGM[t] selected by the composite program selection module 40 based on the composite selection rule may be the same as or different from the system-selectable program sysPGM[s] taken as the (z−1)-th round training-program rnd_trnPGM[z−1], that is, s=t or st.

Please refer to FIG. 10, which is a flowchart showing that the composite program selection module selects the z-th round training-program rnd_trnPGM[z]=sysPGM[t] according to the composite selection rule. First, the training duration-ratio calculation module 401 calculates the program-training duration-ratio rnd_trnDURr[z] of the z-th round according to the z-th program-training duration rnd_trnDUR[z] and the requested daily training duration reqTrnDURpD[usr] of the user (usr) in the doctor's orders (step S501). Then, the composite program selection module 40 initializes the count value of the counter 115 representing the y-th system-selectable program sysPGM[y] to 1 (step S503).

Next, the level adjustment module 403 selectively adjusts the program execution level sysPGM_curLVL[sysPGM[y]] of the system-selectable program sysPGM[y] (step S504). Please refer to the description with reference to FIG. 11 for the details of step S504 performed by the level adjustment module 403.

Further, the training-volume calculation module 41 calculates the y-th program expected training-volume pgm_expVOL(z, sysPGM[y]) of the z-th round, supposing that the system-selectable program sysPGM[y] is selected as the z-th round training-program rnd_trnPGM[z] for the z-th program-training duration rnd_trnDUR[z] (step S505). Please refer to the description with reference to FIG. 12 for the details of step S505 performed by the training-volume calculation module 41.

According to concepts of the present disclosure, the day's remaining training-volume rnd_balVOL[z−1] of the (z−1)-th round in the vector format is viewed as one point in the vector space; and the y-th program expected training-volume pgm_expVOL(z, sysPGM[y]) of the z-th round in the vector format is viewed as another point in the vector space. The vector space distance between the two points in the vector space can be calculated based on the vector distance formula. In step S507, the vector distance calculation module 409 calculates the vector space distance ΔvecD_exp2balVOL(z, sysPGM[y]) between the y-th program expected training-volume pgm_expVOL(z, sysPGM[y]) of the z-th round and the day's remaining training-volume rnd_balVOL[z−1] of the (z−1)-th round according to the vector distance formula by supposing that the system-selectable program sysPGM[y] is selected as the z-th round training-program rnd_trnPGM[z] in the z-th round.

Subsequently, the composite program selection module 40 determines whether the count value of the counter 115 representing the y-th system-selectable program sysPGM[y] is equal to Y (step S509). If the determination result of step S509 is negative, the counter 115 counts up (step S511), and the process repeats step S504. If the determination result of step S509 is positive, it means that the calculation of the vector space distance Δ vecD_exp2balVOL(z, sysPGM[1])˜Δ vecD_exp2balVOL(z, sysPGM[Y]) for all of the system-selectable programs sysPGM[1]˜sysPGM[Y] has been completed. Afterwards, the vector distance comparison module 408 compares the lengths of the vector space distances Δ vecD_exp2balVOL(z, sysPGM[1])˜Δ vecD_exp2balVOL(z, sysPGM[Y]) corresponding to the system-selectable programs sysPGM[1]˜sysPGM[Y] (step S513).

Subsequently, the vector distance comparison module 408 selects the system-selectable program sysPGM[t] among the system-selectable programs sysPGM[1]˜sysPGM[Y] as the z-th round training-program rnd_trnPGM[z] wherein the system-selectable program sysPGM[t] has the shortest vector space distance min {Δ vecD_exp2balVOL(z, sysPGM[1]), . . . , Δ vecD_exp2balVOL(z, sysPGM[Y])} relative to the day's remaining training-volume rnd_balVOL[z−1] of the (z−1)-th round (step S515), that is, t=(argmin (Δ vecD_exp2balVOL((z−1), sysPGM[Y]))).

In short, among the system-selectable programs sysPGM[1]˜sysPGM[Y], the system-selectable program sysPGM[t] having the shortest vector space distance min {Δ vecD_exp2balVOL(z, sysPGM[1]), . . . , Δ vecD_exp2balVOL(z, sysPGM[Y])} can be regarded as the system-selectable program which most likely returns the day's remaining training-volume rnd_balVOL[z] of the z-th round to zero when the user (usr) performs the z-th round of program training. In other words, if the composite program selection module 40 selects and provides the system-selectable program sysPGM[t] for the user (usr) to perform the z-th round of program training, it is easier to achieve the target daily training-volume tgtVOLpD[usr] in that day.

According to concepts of the present disclosure, each system-selectable program sysPGM[1]˜sysPGM[Y] corresponds to one program execution level sysPGM_curLVL[sysPGM[1]]˜sysPGM_curLVL[sysPGM[Y]]. The program execution level sysPGM_curLVL[sysPGM[1]]˜sysPGM_curLVL[sysPGM[Y]] represents the difficulty of the system-selectable program sysPGM[1]˜sysPGM[Y]. Further, the program execution levels sysPGM_curLVL[sysPGM[1]]˜sysPGM_curLVL[sysPGM[Y]] corresponding to the system-selectable programs sysPGM[1]˜sysPGM[Y] are all placed between the system program lowest level sys_minLVL (for example, sys_minLVL=1) and the system program highest level sys_maxLVL (for example, sys_maxLVL=5).

Please refer to FIG. 11, which is a flowchart showing that the level adjustment module selectively adjusts the program execution levels sysPGM_curLVL[sysPGM[1]]˜sysPGM_curLVL[sysPGM[Y]] corresponding to the system-selectable programs sysPGM[1]˜sysPGM[Y]. Please refer to both FIGS. 9 and 11.

First, the level adjustment module 403 sets the counter 115 representing the y-th system selected program to 1 (y=1) (step S401).

Next, the level adjustment module 403 transmits the value of the variable s, the count value of the counter 115 representing the y-th system-selectable program sysPGM[y] to the program similarity calculation module 118 or the storage device 13. The program similarity calculation module 118 calculates (for example, set a=s, b=y) and obtains the program similarity degree pgmSIM(s, y) between the (z−1)-th round training-program rnd_trnPGM[z−1]=sysPGM[s] and the system-selectable program sysPGM[y] (step S403). Alternatively, the level adjustment module 403 looks up the program similarity degree pgmSIM(s, y) between the (z−1)-th round training-program rnd_trnPGM[z−1]=sysPGM[s] and the system-selectable program sysPGM[y] in the lookup table in the storage device 13. Please refer to the description with reference to FIG. 6 for the process of calculating the program similarity degree pgmSIM(s, y), and it is not described in detail herein.

As shown in FIG. 9, the level adjustment module 403 receives the program similarity threshold pgm_simTH (for example, pgm_simTH=0.8) from the storage device 13. In actual applications, the value of the program similarity threshold pgm_simTH could be adjusted according to the situation and is not limited to this example.

In step S405, the level adjustment module 403 compares the program similarity threshold pgm_simTH and the program similarity degree pgmSIM(s, y). If the program similarity degree pgmSIM(s, y) is smaller than or equal to the program similarity threshold pgm_simTH (that is, pgmSIM(s, y)≤pgm_simTH), the determination result of step S405 is NO. This case means that the program similarity degree pgmSIM(s, y) between the system-selectable program sysPGM[y] and the (z−1)-th round training-program rnd_trnPGM[z]=sysPGM[s] is lower. Therefore, the level adjustment module 403 does not need to adjust the program execution level sysPGM_curLVL[sysPGM[y]] of the system-selectable program sysPGM[y].

Otherwise, if the program similarity degree pgmSIM(s, y) is greater than the program similarity threshold pgm_simTH (that is, pgmSIM(s, y)>pgm_simTH), the determination result of step S405 is positive. This case means that the program similarity degree pgmSIM(s, y) between the system-selectable program sysPGM[y] and the (z−1)-th round training-program rnd_trnPGM[z−1]=sysPGM[s] is higher. At this time, when the level adjustment module 403 decides, in the z-th round, to select the system-selectable program sysPGM[y] as the z-th round training-program rnd_trnPGM[z]=sysPGM[y] after considering the user performance in the (z−1)-th round of program training, the program training-volume adjustment parameter pgm_vsclPARM[sysPGM[y]] corresponding to the system-selectable program sysPGM[y] should be increased, decreased, or remained unchanged (step S407).

Step S407 further includes the following steps:

The level adjustment module 403 determines whether the training-program completion rate rnd_pgmCompPCT[z−1] of the (z−1)-th round is greater than or equal to the upgrade threshold of program completion pgm_upTH (rnd_pgmCompPCT[z−1]≥pgm_upTH) (step S407a). If the determination result of step S407a is positive, it means that the training-program completion rate rnd_pgmCompPCT[z−1] of the (z−1)-th round is high when the user (usr) uses the system-selectable program sysPGM[s] as the (z−1)-th round training-program rnd_trnPGM[z−1] in the (z−1)-th round. At this time, for encouraging the user (usr) to further challenge the system-selectable program sysPGM[y] with high program similarity degree pgmSIM(s, y)=pgmSIM(y, s) to the system-selectable program sysPGM[s], the level adjustment module 403 attempts to upgrade the program execution level sysPGM_curLVL[sysPGM[y]] of the system-selectable program sysPGM[y] with high program similarity degree pgmSIM(s, y)=pgmSIM(y, s) to the system-selectable program sysPGM[s]. It is equivalent to increasing the difficulty of the system-selectable program sysPGM[y] with high program similarity degree pgmSIM(s, y)=pgmSIM(y, s) to the system-selectable program sysPGM[s].

In this condition, the level adjustment module 403 further determines whether the program execution level sysPGM_curLVL[sysPGM[y]] of the system-selectable program sysPGM[y] is lower than the system program highest level sys_maxLVL (for example, sys_maxLVL=5) (step S407c). If the determination result of step S407c is negative, it means that the level adjustment module 403 cannot further raise the program execution level sysPGM_curLVL[sysPGM[y]] of the system-selectable program sysPGM[y]. Otherwise, if the determination result of step S407c is positive, the level adjustment module 403 will raise the program execution level sysPGM_curLVL[sysPGM[y]] of the system-selectable program sysPGM[y] (sysPGM_curLVL[sysPGM[y]]++) (step S407e).

If the determination result of step S407a is negative, the level adjustment module 403 further determines whether the training-program completion rate rnd_pgmCompPCT[z−1] of the (z−1)-th round is smaller than the downgrade threshold of program completion pgm_dnTH (rnd_pgmCompPCT[z−1]<pgm_dnTH) (step S407g). If the determination result of step S407g is negative, the level adjustment module 403 does not adjust the program execution level sysPGM_curLVL[sysPGM[y]] of the system-selectable program sysPGM[y].

If the determination result of step S407g is positive, it means that the training-program completion rate rnd_pgmCompPCT[z−1] of the (z−1)-th round is low when the user (usr) uses the system-selectable program sysPGM[s] as the training-program pgm_trnPGM[z−1] in the (z−1)-th round. At this time, in order to prevent the user (usr) from being frustrated by the system-selectable program sysPGM[y] with high program similarity degree pgmSIM(s, y)=pgmSIM(y, s) to the system-selectable program sysPGM[s] and interrupting the training, the level adjustment module 403 will attempt to downgrade the program execution level sysPGM_curLVL[sysPGM[y]] of the system-selectable program sysPGM[y] with high program similarity degree pgmSIM(s, y)=pgmSIM(y, s) to the system-selectable program sysPGM[s]. It is equivalent to decreasing the difficulty of the system-selectable program sysPGM[y] with high program similarity degree pgmSIM(s, y)=pgmSIM(y, s) to the system-selectable program sysPGM[s].

In this condition, the level adjustment module 403 further determines whether the program execution level sysPGM_curLVL[sysPGM[y]] of the system-selectable program sysPGM[y] is higher than the system program lowest level sys_minLVL (for example, sys_minLVL=1) (step S407i). If the determination result of step S407i is negative, it means that the level adjustment module 403 cannot further lower the program execution level sysPGM_curLVL[sysPGM[y]] of the system-selectable program sysPGM[y]. Otherwise, if the determination result of step S407i is positive, the level adjustment module 403 will lower the program execution level sysPGM_curLVL[sysPGM[y]] of the system-selectable program sysPGM[y] (sysPGM_curLVL[sysPGM[y]]−−) (step S407k).

According to concepts of the present disclosure, when the training-program completion rate rnd_pgmCompPCT[z−1] of the (z−1)-th round is high, it means that when the training system tmnSYS selects the system-selectable program sysPGM[s] in the (z−1)-th round, the program training has a good effect on the user (usr). Therefore, the training system tmnSYS can raise the program execution level sysPGM_curLVL[sysPGM[y]] of other system-selectable programs with higher program similarity to the system-selectable program sysPGM[s], thereby encouraging the user (usr) to challenge more difficult levels.

Otherwise, when the training-program completion rate rnd_pgmCompPCT[z−1] of the (z−1)-th round is low, it means that when the training system trnSYS selects the system-selectable program sysPGM[s] in the (z−1)-th round, the program training has a poor effect on the user (usr). At this time, in order to prevent the user from being frustrated and abandoning the use of the training system trnSYS, the training system trnSYS can lower the program execution level sysPGM_curLVL[sysPGM[y]] of other system-selectable programs with higher program similarity to the system-selectable program sysPGM[s]. That is, the difficulty of the related system-selectable program is lowered to improve the sense of achievement of the user (usr). Accordingly, the training system trnSYS of the present disclosure can prevent the user (usr) from getting frustrated due to a low completion rate and then losing the willingness to perform the training.

In actual applications, the actual values of the upgrade threshold of program completion pgm_upTH and the downgrade threshold of program completion pgm_dnTH need not be limited, for example, the upgrade threshold of program completion pgm_upTH=0.7 and the downgrade threshold of program completion pgm_dnTH=0.5. However, the upgrade threshold of program completion pgm_upTH should be greater than the downgrade threshold of program completion pgm_dnTH, that is, pgm_upTH>pgm_dnTH.

In short, in step S407, if the user (usr) performs well in the (z−1)-th round of program training, the level adjustment module 403 raises the program execution level sysPGM_curLVL[sysPGM[y]] of the system-selectable program sysPGM[y], thereby raising the program training-volume adjustment parameter pgm_vsclPARM[sysPGM[y]] corresponding to the system-selectable program sysPGM[y]. If the program execution level sysPGM_curLVL[sysPGM[y]] of the system-selectable program sysPGM[y] is raised, it means that the difficulty of the system-selectable program sysPGM[y] increases. At this time, the expected training-volume calculation module 407 will calculate and obtain a greater value for the expected training-volume pgm_expVOL(z,sysPGM[y]) corresponding to the system-selectable program sysPGM[y].

Otherwise, in step S407, if the user (usr) performs poorly in the (z−1)-th round of program training, the level adjustment module 403 lowers the program execution level sysPGM_curLVL[sysPGM[y]] of the system-selectable program sysPGM[y], thereby lowering the program training-volume adjustment parameter pgm_vsclPARM[sysPGM[y]] corresponding to the system-selectable program sysPGM[y]. If the program execution level sysPGM_curLVL[sysPGM[y]] of the system-selectable program sysPGM[y] is lowered, it means that the difficulty of the system-selectable program sysPGM[y] decreases. At this time, the expected training-volume calculation module 407 will calculate and obtain a smaller value for the expected training-volume pgm_expVOL(z,sysPGM[y]) corresponding to the system-selectable program sysPGM[y].

Further, in step S407, if the user (usr) performs averagely in the (z−1)-th round of program training, the level adjustment module 403 maintains the program execution level sysPGM_curLVL[sysPGM[y]] of the system-selectable program sysPGM[y]. At this time, the difficulty of the system-selectable program sysPGM[y] is unchanged, and when the expected training-volume calculation module 407 calculates the expected training-volume pgm_expVOL(z,sysPGM[y]) corresponding to the system-selectable program sysPGM[y], the value will not become greater or smaller.

After step S407, the level adjustment module 403 determines whether the value of the counter 115 representing the y-th system-selectable program sysPGM[y] is equal to Y (step S409). If the determination result of step S409 is positive, the process ends. If the determination result of step S409 is negative, the level adjustment module 403 makes the counter 115 count up (step S411) and then repeats step S403.

Before the user (usr) first operates the training system trnSYS, the program execution levels sysPGM_curLVL[sysPGM[1]]˜sysPGM_curLVL[sysPGM[Y]] could be preset to the system program lowest level sys_minLVL (for example, sysPGM_curLVL[sysPGM[1]]= . . . sysPGM_curLVL[sysPGM[Y]]=sys_minLVL=1) available in the training system trnSYS. After one day of training, when the user (usr) uses the training system trnSYS again, the program execution levels sysPGM_curLVL[sysPGM[1]]˜sysPGM_curLVL[sysPGM[Y]] have been updated (upgraded, downgraded or unchanged) after one day's operation (Z rounds of program training in total). At this time, the system-selectable programs sysPGM[1]˜sysPGM[Y] in the training system trnSYS will adopt the program execution levels sysPGM_curLVL[sysPGM[1]]˜sysPGM_curLVL[sysPGM[Y]] previously stored in the training system trnSYS.

As described above, the selection strategy judgment module 111a may select the component for generating the variable t according to different conditions. According to concepts of the present disclosure, if the selection strategy judgment module 111a selects the random number generator 111c or the heterogeneous program selection module 111e to generate the variable t, the values of the program execution levels sysPGM_curLVL[sysPGM[1]]˜sysPGM_curLVL[sysPGM[Y]] corresponding to the system-selectable programs sysPGM[1]˜sysPGM[Y] keep unchanged. Alternatively, if the selection strategy judgment module 111a selects the composite program selection module 40 to generate the variable t, the level adjustment module 403 in the composite program selection module 40 will increase, decrease, or maintain the values of the program execution levels sysPGM_curLVL[sysPGM[1]]˜sysPGM_curLVL[sysPGM[Y]] corresponding to the system-selectable programs sysPGM[1]˜sysPGM[Y] as the user (usr) performs the training.

Please refer to FIG. 12, which is a flowchart showing that the training-volume calculation module calculates the y-th program expected training-volume pgm_expVOL(z, sysPGM[y]) of the z-th round corresponding to the system-selectable program sysPGM[y] when the system-selectable program sysPGM[y] is selected as the z-th round training-program rnd_trnPGM[z]=sysPGM[y] for the z-th program-training duration. FIG. 12 further illustrates step S505 of FIG. 10.

Please refer to both FIGS. 9 and 12. It can be seen from FIG. 9 that the training-volume calculation module 41 includes: the original training-volume calculation module 405, the training-volume adjustment parameter calculation module 406, and the expected training-volume calculation module 407.

First, the original training-volume calculation module 405 multiplies the program-training duration-ratio rnd_trnDURr[z] of the z-th round and the program training-aspect contributing effect pgm_allASP[sysPGM[y]] of the system-selectable program sysPGM[y], and the product is used as the y-th program original training-volume pgm_origVOL(z, sysPGM[y]) of the z-th round corresponding to the system-selectable program sysPGM[y] (step S505a), for example, pgm_origVOL(z, sysPGM[y])=rnd_trnDURr[z]×pgm_allASP[sysPGM[y]].

Referring to the calculation made by the original training-volume calculation module 405 to calculate the y-th program original training-volume pgm_origVOL(z, sysPGM[y]) of the z-th round corresponding to the system-selectable program sysPGM[y], it can be seen that, the y-th program original training-volumes pgm_origVOL(z, sysPGM[y]) of the z-th round corresponding to the system-selectable program sysPGM[y] is equivalent to the maximum training-volume that the system-selectable program sysPGM[y] can provide under the duration-ratio of the z-th program-training duration rnd_trnDUR[z] to the requested daily training duration reqTmnDURpD[usr] (for example, 10 minutes out of the 30 minutes), wherein it is supposed that the system-selectable program sysPGM[y] is selected as the z-th round training-program rnd_trnPGM[z]=sysPGM[y]. Or, the y-th program original training-volume pgm_origVOL(z, sysPGM[y]) of the z-th round corresponding to the system-selectable program sysPGM[y] can be referred as the full training-volume wherein the system-selectable program sysPGM[y] is selected as the z-th round training-program rnd_trnPGM[z]=sysPGM[y] for the z-th program-training duration rnd_trnDUR[z].

Following this example, the program-training duration-ratio rnd_trnDURr[z] of the z-th round could be expressed as 10 minutes/30 minutes. Based on the z-th-round program-training duration-ratio rnd_trnDURr[z]=10/30 and the program contributing training-aspect effects pgm_allASP[sysPGM[1]]˜pgm_allASP[sysPGM[3]], it can be further deduced that the system-selectable program sysPGM[1] can provide the program original training-volume pgm_origVOL(z, sysPGM[1])=rnd_trnDURr[z]*pgm_allASP[sysPGM[1]]=10/30*[25, 40, 25, 10, 0]=[8.3, 10.3, 8.3, 3.3, 0] over the z-th program-training duration rnd_trnDUR[z]; the system-selectable program sysPGM[2] can provide the program original training-volume pgm_origVOL(z, sysPGM[2])=rnd_trnDURr[z]*pgm_allASP[sysPGM[2]]=10/30*[60, 30, 0, 10, 0]=[20, 10, 0, 3.3, 0] over the z-th program-training duration rnd_trnDUR[z]; and the system-selectable program sysPGM[3] can provide the program original training-volume pgm_origVOL(z, sysPGM[3])=rnd_trnDURr[z]*pgm_allASP[sysPGM[3]]=10/30*[30, 0, 0, 0, 70]=[10, 0, 0, 0, 23.3] over the z-th program-training duration rnd_trnDUR[z].

Further, the training-volume adjustment parameter calculation module 406 receives the program training-volume adjustment base pgm_vsclBASE[sysPGM[y]] corresponding to the system-selectable program sysPGM[y] from the storage device 13, and receives the program execution level sysPGM_curLVL[sysPGM[y]], upgraded after the z-th round, of the system-selectable program sysPGM[y] from the level adjustment module 403. Then, the training-volume adjustment parameter calculation module 406 calculates the program training-volume adjustment parameter pgm_vsclPARM[sysPGM[y]] of the system-selectable program sysPGM[y] according to the program training-volume adjustment base pgm_vsclBASE and the program execution level sysPGM_curLVL[sysPGM[y]] of the system-selectable program sysPGM[y] (step S505c), for example, pgm_vsclPARM[sysPGM[y]]=pgm_vsclBASE[sysPGM[y]]^{sysPGM_curLVL[sysPGM[y]]}.

According to concepts of the present disclosure, the program training-volume adjustment base pgm_vsclBASE[sysPGM[y]] is a variable for increasing the expected training-volume pgm_expVOL(z, sysPGM[y]) corresponding to the system-selectable program sysPGM[y]. Normally, the program training-volume adjustment base pgm_vsclBASE[sysPGM[y]] will be set to a value greater than 1 (pgm_vsclBASE[sysPGM[y]]>1). How to set and adjust the values of the program training-volume adjustment bases pgm_vsclBASE[sysPGM[1]]˜pgm_vsclBASE[sysPGM[Y]] is related to variations of the applications, and a detailed description is not given herein.

In actual applications, the system-selectable program sysPGM[y] with a higher difficulty can be set to have a greater program training-volume adjustment base pgm_vsclBASE[sysPGM[y]] (for example, 1.5). Similarly, the system-selectable program sysPGM[y] with a lower difficulty can be set to have a smaller program training-volume adjustment base pgm_vsclBASE[sysPGM[y]] (for example, 1.1).

Alternatively, for simplifying the calculations, the training system trnSYS can set the program training-volume adjustment bases pgm_vsclBASE[sysPGM[1]]˜pgm_vsclBASE[sysPGM[Y]] of the system-selectable programs sysPGM[1]˜sysPGM[Y] to the same value. For example, all of the program training-volume adjustment bases pgm_vsclBASE[sysPGM[1]]˜pgm_vsclBASE[sysPGM[Y]] are equal to the preset training-volume adjustment base sys_dfltBASE=1.2 of the training system trnSYS.

For further simplifying the calculations, the training system trnSYS can further set all of the program training-volume adjustment bases pgm_vsclBASE[sysPGM[1]]˜pgm_vsclBASE[sysPGM[Y]] of the system-selectable program sysPGM[1]˜sysPGM[Y] to 1. In such applications, the program training-volume adjustment parameter pgm_vsclPARM[sysPGM[y]] is equal to 1. In this case, the training-volume adjustment parameter calculation module 406 and the level adjustment module 403 could be further omitted. Based on this supposition, the y-th program original training-volume pgm_origVOL(z, sysPGM[y]) of the z-th round corresponding to the system-selectable program sysPGM[y] is identical to the y-th program expected training-volume pgm_expVOL(z, sysPGM[y]) of the z-th round corresponding to the system-selectable program sysPGM[y], that is, pgm_origVOL(z, sysPGM[y])=pgm_expVOL(z, sysPGM[y]).

After step S505a, the original training-volume calculation module 405 transmits the y-th program original training-volume pgm_origVOL(z, sysPGM[y]) of the z-th round corresponding to the system-selectable program sysPGM[y] to the expected training-volume calculation module 407. After step S505c, the training-volume adjustment parameter calculation module 406 transmits the program training-volume adjustment parameter pgm_vsclPARM[sysPGM[y]] of the system-selectable program sysPGM[y] to the expected training-volume calculation module 407. The sequence of steps S505a and S505c is not limited. Alternatively, steps S505a and S505c could be performed simultaneously.

Subsequently, the expected training-volume calculation module 407 calculates the y-th program expected training-volumes pgm_expVOL(z, sysPGM[y]) of the z-th round corresponding to the system-selectable program sysPGM[y] according to the program training-volume adjustment parameter pgm_vsclPARM[sysPGM[y]] and the program original training-volume pgm_origVOL(z, sysPGM[y]) of the system-selectable program sysPGM[y] (step S505e), for example, pgm_expVOL(z, sysPGM[y])=pgm_vsclPARM[sysPGM[y]] x pgm_origVOL(z, sysPGM[y]).

As described above, the program training-aspect contributing effect pgm_allASP[sysPGM[y]] could be represented in a vector format. Therefore, the calculations in steps S505a and S505e of FIG. 12 could be performed based on the vector format. For example, the y-th program original training-volumes pgm_origVOL(z, sysPGM[y]) of the z-th round corresponding to the system-selectable program sysPGM[y] calculated by and obtained from the original training-volume calculation module 405 based on the program training-aspect contributing effect pgm_allASP[sysPGM[y]] of the system-selectable program sysPGM[y] could be expressed as a vector containing X values corresponding to X training aspects trnASP[1]˜trnASP[X]. Similarly, the y-th program expected training-volume pgm_expVOL(z, sysPGM[y]) of the z-th round calculated by and obtained from the expected training-volume calculation module 407 based on the y-th program original training-volume pgm_origVOL(z, sysPGM[y]) of the z-th round corresponding to the system-selectable program sysPGM[y] could be expressed as a vector containing X values corresponding to X training aspects trnASP[1]˜trnASP[X].

As described above, the program selection module 111, according to concepts of the present disclosure, selects one of the random number generator 111c, the heterogeneous program selection module 111e, and the composite program selection module 40 to select the z-th round training-program rnd_trnPGM[z]=sysPGM[t] in response to the value of z (z=1 or z>1) and the training-duration concentration level rnd_durConcPCT[z−1] of the (z−1)-th round of the user (usr). No matter which one of the random number generator 111c, the heterogeneous program selection module 111e, and the composite program selection module 40 is selected by the program selection module 111 to perform the selection of the z-th round training-program rnd_trnPGM[z]=sysPGM[t], the program execution module 113 will execute the content of the system-selectable program sysPGM[t] in the z-th round according to the selection result given by the program selection module 111. Thus, the program execution module 113 uses the program-play device 17 (screen, speaker, and the like) to output images, sounds, and the like of the system-selectable program sysPGM[t].

After the program execution module 113 completes the z-th round of program training, the concentration calculation module 116 calculates and obtains the training-duration concentration level rnd_durConcPCT[z] of the z-th round according to the sensing results obtained from the sensing device 19 within the z-th program-training duration mnd_trnDUR[z]. Then, the program completion calculation module 117 calculates and obtains the training-program completion rate rnd_pgmCompPCT[z] of the z-th round according to the execution results (for example, scoring result) of the program execution module 113 executing the z-th round training-program rnd_trnPGM[z]=sysPGM[t]. Afterward, the training result calculation module 50 performs analysis and calculation on the training results, as shown in FIGS. 13 and 14.

Please refer to FIG. 13, which is a block diagram illustrating a training result calculation module. The training result calculation module 50 includes: a participation factor calculation module 501, an actual training-volume calculation module 503, and a remaining training-volume calculation module 505.

The participation factor calculation module 501 receives the training-program completion rate md_pgmCompPCT[z] of the z-th round from the program completion calculation module 117, and receives the training-duration concentration level rnd_durConcPCT[z] from the concentration calculation module 116. Then, the participation factor calculation module 501 calculates and generates the user participation factor rndUsr_partFA[z] of the z-th round based on the training-program completion rate rnd_pgmCompPCT[z] of the z-th round and the training-duration concentration level rnd_durConcPCT[z] of the z-th round, for example,

$$rndUsr\_partFA [ z ] = \frac{rnd\_durConcPCT [ z ] + rnd\_pgmCompPCT [ z ]}{2} or$$

$$rndUsr\_partFA [ z ] = \sqrt{rnd\_durConcPCT [ z ] \times rnd\_pgmCompPCT [ z ]} .$$

The actual training-volume calculation module 503 receives the t-th program expected training-volume pgm_expVOL(z, sysPGM[t]) of the z-th round from the expected training-volume calculation module 407, and receives the user participation factor rndUsr_partFA[z] of the z-th round from the participation factor calculation module 501. Then, the actual training-volume calculation module 503 calculates and generates the user actual training-volume rndUsr_realVOL[z] of the z-th round based on the t-th program expected training-volumes pgm_expVOL(z, sysPGM[t]) of the z-th round and the user participation factor rndUsr_partFA[z], for example, rndUsr_realVOL[z]=rndUsr_partFA[z] x pgm_expVOL(z, sysPGM[t]). As described above, the t-th program expected training-volume pgm_expVOL(z, sysPGM[t]) of the z-th round calculated by and obtained from the expected training-volume calculation module 407 includes a vector containing X values corresponding to the X training aspects trnASP[1]˜tmnASP[X]. Consequently, the user actual training-volume rndUsr_realVOL[z] of the z-th round, calculated by the actual training-volume calculation module 503 based on the t-th program expected training-volume pgm_expVOL(z, sysPGM[t]) of the z-th round in the vector format, also has a vector format.

The remaining training-volume calculation module 505 receives the day's remaining training-volume rnd_balVOL[z−1] of the (z−1)-th round from the storage device 13, and receives the user actual training-volume rndUsr_realVOL[z] of the z-th round from the actual training-volume calculation module 503. The day's remaining training-volume rnd_balVOL[z−1] of the (z−1)-th round is calculated by and obtained from the remaining training-volume calculation module 505 after the (z−1)-th program-training duration, and is temporarily stored in the storage device 13. Subsequently, the remaining training-volume calculation module 505 calculates and generates the day's remaining training-volume rnd_balVOL[z] of the z-th round based on the day's remaining training-volume rnd_balVOL[z−1] of the (z−1)-th round and the user actual training-volume rndUsr_realVOL[z] of the z-th round, example, rnd_balVOL[z]=rnd_balVOL[z−1]-rndUsr_realVOL[z] Similarly, the day's remaining training-volume rnd_balVOL[z] of the z-th round, calculated by and obtained from the remaining training-volume calculation module 505, is stored in the storage device 13 as the calculation basis for the remaining training-volume calculation module 505 after the (z+1)-th program-training duration.

According to concepts of the present disclosure, if any of the X values included in the day's remaining training-volume rnd_balVOL[z] of the z-th round and corresponding to the X training aspects trnASP[1]˜trnASP[X] is a negative value, the value is reset to 0. For example, in case of the (z−1)-th-round day's remaining training-volume rnd_balVOL[z−1]=[6, 15.5, 20, 12, 15] and the z-th-round user actual training-volume rndUsr_realVOL[z]=[6.6, 10.5, 6.6, 2.6, 0], the day's remaining training-volume rnd_balVOL[z] of the z-th round calculated based on the equation rnd_balVOL[z]=rnd_balVOL[z−1]−rndUsr_realVOL[z] results in rnd_balVOL[z]=[6, 15.5, 20, 12, 15]-[6.6, 10.5, 6.6, 2.6, 0]=[−0.6, 5, 13.4, 9.4, 15]. At this time, the training system trnSYS modifies the day's remaining training-volume rnd_balVOL[z] of the z-th round to rnd_balVOL[z]=[0, 5, 13.4, 9.4, 15].

In the case of z=1, the day's remaining training-volume rnd_balVOL[z−1] of the z-th round is equal to the target daily training-volume tgtVOLpD[usr]. Accordingly, after the first round (z=1) of program training, the day's remaining training-volume rnd_balVOL[1] corresponding to the first round (z=1) is equal to the difference between the target daily training-volume tgtVOLpD[usr] and the user actual training-volume rndUsr_realVOL[1] of the first round (z=1), for example, rnd_balVOL[z]=tgtVOLpD[usr]−rndUsr_realVOL[1]. As described above, the target daily training-volume tgtVOLpD[usr] of the user (usr) could be expressed in the vector format, and the user actual training-volume rndUsr_realVOL[z] could also be expressed in the vector format. Therefore, the remaining training-volume calculation module 505 calculates the day's remaining training-volume rnd_balVOL[z] corresponding to the z-th round vectorially, and the day's remaining training-volume rnd_balVOL[z] corresponding to the z-th round is also in the vector format.

It can be seen from further analysis of the calculation of the day's remaining training-volume rnd_balVOL[z] of the z-th round that the calculation of the day's remaining training-volume rnd_balVOL[z] of the z-th round involves various parameters, including: the target daily training-volume tgtVOLpD[usr] of the user (usr), the length of the z-th program-training duration rnd_trnDUR[z], the requested daily training duration reqTmnDURpD[usr] of the user (usr), the program training-aspect contributing effect pgm_allASP[sysPGM[t]] of the z-th round training-program rnd_trnPGM[z]=sysPGM[t], the training-duration concentration level rnd_durConcPCT[z] of the z-th round of the user (usr) within the z-th program-training duration rnd_trnDUR[z] and the training-program completion rate rnd_pgmCompPCT[z] of the z-th round. These parameters related to the day's remaining training-volume rnd_balVOL[z] of the z-th round not only involve the requirements of the doctor's orders, the effectiveness of the training-programs, and the length of the training-program, but also take into account the personal state of the user (usr) performing the training.

Please refer to FIG. 14, which is a flowchart showing how the training result calculation module calculates the day's remaining training-volume rnd_balVOL[z] of the z-th round. Please refer to both FIGS. 13 and 14.

First, the expected training-volume calculation module 407 calculates the t-th program expected training-volume pgm_expVOL(z, sysPGM[t]) of the z-th round corresponding to the system-selectable program sysPGM[y] wherein the system-selectable program sysPGM[y] is used in the z-th round as the z-th round training-program (step S71). Next, the training result calculation module 50 calculates the user participation factor rndUsr_partFA[z] of the z-th round (step S73).

Please refer to both FIGS. 13 and 14. Step S73 further includes the following steps:

The concentration calculation module 116 calculates the training-duration concentration level rnd_durConcPCT[z] of the z-th round of the user (usr) according to the sensing results obtained from the sensing device 19 (step S73a). The program completion calculation module 117 calculates the training-program completion rate rnd_pgmCompPCT[z] of the z-th round of the user (usr) according to the execution results (for example, the score, accuracy, reaction time, and the like of the user (usr)) of the program execution device 11 in the z-th round (step S73c). Further, the participation factor calculation module 501 calculates the user participation factor rndUsr_partFA[z] of the z-th round according to the training-duration concentration level rnd_durConcPCT[z] of the z-th round of the user (usr) and the training-program completion rate rnd_pgmCompPCT[z] of the z-th round (step S73e).

The training-duration concentration level mnd_durConcPCT[z] of the z-th round and the training-program completion rate rnd_pgmCompPCT[z] of the z-th round of the user (usr) ranges between 0 and 1. In the embodiments of the present disclosure, it can be supposed that the user participation factor rndUsr_partFA[z] of the z-th round is the arithmetic mean or the geometric mean of the training-duration concentration level rnd_durConcPCT[z] of the z-th round and the training-program completion rate rnd_pgmCompPCT[z] of the z-th round of the user (usr), that is,

$$rndUsr\_partFA [ z ] = \frac{rnd\_durConcPCT [ z ] + rnd\_pgmCompPCT [ z ]}{2} or$$

$$rndUsr\_partFA [ z ] = \sqrt{rnd\_durConcPCT [ z ] \times rnd\_pgmCompPCT [ z ]} .$$

If the user participation factor rndUsr_partFA[z] of the z-th round is defined as the arithmetic mean or the geometric mean of the training-duration concentration level mnd_durConcPCT[z] of the z-th round and the training-program completion rate rnd_pgmCompPCT[z] of the z-th round of the user (usr), the user participation factor rndUsr_partFA[z] of the z-th round is placed between 0 and 1. In actual applications, the calculation for obtaining the user participation factor rndUsr_partFA[z] of the z-th round is not limited.

After step S73, the actual training-volume calculation module 503 calculates the user actual training-volume rndUsr_realVOL[z] of the z-th round by multiplying the t-th program expected training-volume pgm_expVOL(z, sysPGM[t]) of the z-th round corresponding to the system-selectable program sysPGM[t] and the user participation factor rndUsr_partFA[z] of the z-th round (step S75). If the user participation factor rndUsr_partFA[z] of the z-th round ranges between 0 and 1, it could be deduced that the y-th program expected training-volume pgm_expVOL(z, sysPGM[t]) of the z-th round corresponding to the system-selectable program sysPGM[t] must be greater than or equal to the user actual training-volume rndUsr_realVOL[z] of the z-th round, that is, pgm_expVOL(z, sysPGM[t])≥rndUsr_realVOL[z].

Afterward, the remaining training-volume calculation module 505 further calculates the day's remaining training-volume rnd_balVOL[z] of the z-th round according to the day's remaining training-volume rnd_balVOL[z−1] of the (z−1)-th round and the user actual training-volume rndUsr_realVOL[z] of the z-th round (step S77). According to concepts of the present disclosure, the training result calculation module 50 can store the calculation result of the day's remaining training-volume rnd_balVOL[z] of the z-th round in the storage device 13. Subsequently, after the program execution module 113 completes the (z+1)-th round training-program rnd_trnPGM[z+1], the training result calculation module 50 reads the day's remaining training-volume rnd_balVOL[z] of the z-th round previously temporarily stored in the storage device 13.

In other words, the process of FIG. 14 will be repeated after the first to Z-th program-training durations rnd_trnDUR[1]˜rnd_trnDUR[Z] are completed. That is, after the (z−1)-th program-training duration rnd_trnDUR[z−1], the process calculates the user actual training-volume rndUsr_realVOL[z−1] of the (z−1)-th round and the day's remaining training-volume rnd_balVOL[z−1] of the (z−1)-th round as the calculation basis for the z-th round. Similarly, after the z-th program-training duration rnd_trnDUR[z], the process calculates the user actual training-volume rndUsr_realVOL[z] of the z-th round and the day's remaining training-volume rnd_balVOL[z] of the z-th round as the calculation basis for the (z+1)-th round, and so on.

In the Z-th round (z=Z), the day's remaining training-volume rnd_balVOL[z] of the Z-th round represents the day's remaining training-volume rnd_balVOL[Z] after the user uses the training system trnSYS to perform Z rounds of program training. Although the next round of program training is not needed, the training system trnSYS could still store the day's remaining training-volume rnd_balVOL[Z] of the Z-th round (z=Z) in the storage device 13. These data can be used to analyze the training effects provided by the training system trnSYS or for the doctor to observe the practice condition of the user (usr) after a series of program training. How to use the various training parameters and training results related to the daily training process, which are stored in the storage device 13 by the training system trnSYS for subsequent use, is related to variations of the applications, and a detailed description is not given in the present disclosure.

As described above, the training system trnSYS, according to concepts of the present disclosure, divides the day's training process of the user (usr) into Z rounds. Moreover, the first to Z-th rounds training-programs rnd_trnPGM[1]˜rnd_trnPGM[Z] and the difficulty levels of the training-programs are adjusted after considering the z-th-round training-duration concentration level rnd_durConcPCT[z] and the training-program completion rate rnd_pgmCompPCT[z] of the z-th round of the user (usr) presented in the sensing results obtained in the previous round.

When the selection strategy judgment module 111a determines that the concentration level of the user (usr) within the z-th program-training duration rnd_trnDUR[z] is insufficient according to the calculation results obtained from the concentration calculation module 116, the training system trnSYS determines that the user is not interested in the program type of the z-th round training-program rnd_trnPGM[z]. At this time, the selection strategy judgment module 111a will use the heterogeneous program selection module 111e to intentionally select a different type of system-selectable program sysPGM[y] from the type of the z-th round training-program rnd_trnPGM[z] (that is, low program similarity degree between the system-selectable program sysPGM[y] and the z-th round training-program rnd_trnPGM[z]) to serve as the (z+1)-th round training-program rnd_trnPGM[z+1] for the user (usr) to operate.

When the selection strategy judgment module 111a determines that the concentration level of the user (usr) within the z-th round of program-training duration is sufficient according to the calculation results obtained from the concentration calculation module 116, the selection strategy judgment module 111a will use the composite program selection module 40 to select the (z+1)-th round training-program rnd_trnPGM[z+1] after comprehensively considering various parameters such as the program training-volume adjustment base pgm_vsclBASE[sysPGM[y]], the program execution level sysPGM_curLVL[sysPGM[y]] and the program original training-volume pgm_origVOL(z, sysPGM[y]). Therefore, the training system trnSYS of the present disclosure is an adaptive training system that can provide the most suitable training for the user (usr) in quick response to the physical and mental states of the user (usr).

As described above, by providing the concentration calculation module 116, the program completion calculation module 117 and the program similarity calculation module 118 in the program execution device 11, the program selection module 111, and the training result calculation module 50 can grasp the actual performance of the user (usr) in the training process more accurately. By adjusting each of the first to Z-th rounds training-programs rnd_trnPGM[1]˜rnd_trnPGM[Z], the training system trnSYS can evaluate the training-duration concentration level rnd_durConcPCT[z] and the training-program completion rate rnd_pgmCompPCT[z] of the z-th round of the user (usr) in real-time after each round of program training, and the evaluation will serve as the basis for selecting the next round training-program.

According to concepts of the present disclosure, except that the first round training-program rnd_trnPGM[1] is randomly selected by the training system trnSYS, the other training-programs rnd_trnPGM[2]˜rnd_trnPGM[Z], starting from the second round, are all determined based on the current condition of the user (usr) executing the program. Therefore, the training system trnSYS provided by the present disclosure can select one of the system-selectable programs sysPGM[1]˜sysPGM[Y] which is most suitable for the actual physical and mental states of the user (usr) in a more flexible way according to the user training-aspect contributing ratio usr_allASPr[usr] required in the doctor's orders, so as to encourage the user (usr) to perform the training for the requested daily training duration reqTmnDURpD[usr] as required in the doctor's orders.

Table 6 summarizes how the training-duration concentration level rnd_durConcPCT[z] of the z-th round generated by the concentration calculation module 116, the training-program completion rate rnd_pgmCompPCT[z] of the z-th round generated by the program completion calculation module 117, and the program similarity degree pgmSIM(a, b) between any two system-selectable programs sysPGM[a] and sysPGM[b] generated by the program similarity calculation module 118 affect the program selection module 111 in selecting the programs and affect the training result calculation module 50 in analyzing the training conditions of the user (usr). In Table 6, NA indicates that the calculation results generated by the calculation module will not be used.

TABLE 6

| | | Program similarity | | Program completion |
|---|---|---|---|---|
| | | calculation module | Concentration calculation | calculation module |
| Calculation module | | 118 | module 116 | 117 |
| Calculation results | | program similarity | training-duration | training-program |
| output from the | | degree pgmSIM(a, | concentration level | completion rate |
| calculation module | | b) | rnd_durConcPCT[z − 1] of the | rnd_pgmCompPCT[z − 1] |
| | | a = s, b = y = 1~Y | (z − 1)-th round, | of the (z − 1)-th |
| | | | training-duration | round, |
| | | | concentration level | training-program |
| | | | rnd_durConcPCT[z] of the z- | completion rate |
| | | | th round | rnd_pgmCompPCT[z] |
| | | | | of the z-th round |
| Program | Selection | NA | selecting heterogeneous | NA |
| selection | strategy | | program selection module | |
| module | judgment | | 111e or composite program | |
| 111 | module 111a | | selection module 40 to select | |
| | | | the z-th round training- | |
| | | | program | |
| | | | rnd_trnPGM[z] = sysPGM[t] | |
| | | | according to the comparison | |
| | | | of the training-duration | |
| | | | concentration level | |
| | | | rnd_durConcPCT[z − 1] of the | |
| | | | (z − 1)-th round and preset | |
| | | | concentration threshold | |
| | | | preset_concTH | |
| | Heterogeneous | selecting system- | NA | NA |
| | program | selectable program | | |
| | selection | sysPGM[t] with the | | |
| | module 111e | smallest program | | |
| | | similarity degree | | |
| | | according to sorted | | |
| | | order of program | | |
| | | similarity degrees | | |
| | | pgmSIM(s, 1~Y) | | |
| | Composite | The composite | NA | The composite |
| | program | program selection | | program selection |
| | selection | module 40 | | module 40 |
| | module 40 | selectively adjusts | | selectively adjusts |
| | | the program | | the program |
| | | execution level | | execution level |
| | | sysPGM_curLVL[sysPGM[y]] | | sysPGM_curLVL[sysPGM[y]] |
| | | of the | | of the |
| | | system-selectable | | system-selectable |
| | | program sysPGM[y] | | program sysPGM[y] |
| | | according to the | | according to the |
| | | training-program | | training-program |
| | | completion rate | | completion rate |
| | | rnd_pgmCompPCT[z − 1] | | rnd_pgmCompPCT[z − 1] |
| | | of the (z − 1)-th | | of the (z − 1)-th |
| | | round and the | | round and the |
| | | program similarity | | program similarity |
| | | degree pgmSIM(s, y). | | degree pgmSIM(s, y). |
| | | Also, the calculated | | Also, the calculated |
| | | y-th program | | y-th program |
| | | expected training- | | expected training- |
| | | volume | | volume |
| | | pgm_expVOL(z, | | pgm_expVOL(z, |
| | | sysPGM[y]) of the z- | | sysPGM[y]) of the z- |
| | | th round is changed. | | th round is changed. |
| | | And, the vector | | And, the vector |
| | | space distance | | space distance |
| | | ΔvecD_exp2balVOL | | ΔvecD_exp2balVOL |
| | | (z, sysPGM[y]) | | (z, sysPGM[y]) |
| | | between the y-th | | between the y-th |
| | | program expected | | program expected |
| | | training-volume | | training-volume |
| | | pgm_expVOL(z, | | pgm_expVOL(z, |
| | | sysPGM[y]) of the z- | | sysPGM[y]) of the z- |
| | | th round and the | | th round and the |
| | | day's remaining | | day's remaining |
| | | training-volume | | training-volume |
| | | rnd_balVOL[z − 1] of | | rnd_balVOL[z − 1] of |
| | | the (z − 1)-th round is | | the (z − 1)-th round is |
| | | also changed, | | also changed, |
| | | thereby affecting the | | thereby affecting the |
| | | selection of the z-th | | selection of the z-th |
| | | round training- | | round training- |
| | | program | | program |
| | | rnd_trnPGM[z] = | | rnd_trnPGM[z] = |
| | | sysPGM[t]. | | sysPGM[t]. |

| | | |
|---|---|---|
| Training result | NA | The user participation factor rndUsr_partFA[z] of the |
| calculation module 50 | | z-th round is calculated according to the training- |

duration concentration level rnd_durConcPCT[z] of
the z-th round and the training-program completion
rate rnd_pgmCompPCT[z] of the z-th round.
The user actual training-volume rndUsr_realVOL[z]
of the z-th round varies with the user participation
factor rndUsr_partFA[z] of the z-th round, and the
day's remaining training-volume rnd_balVOL[z] of
the z-th round is also changed.

The program execution method proposed in the present disclosure can be applied to various computer program products. The computer program product stores thereon the software program, and the software program executes the program execution method of the present disclosure.

Those of ordinary skill in the art should understand that in the above description, the various logical blocks, modules, circuits, and steps taken as examples can be implemented by electronic hardware, computer software, or a combination thereof. The connections, regardless of being expressed as signal connections, connections, communication, coupling, electrical connections, or the like, represent that signal/data/information exchange or transmission for implementing the logical blocks, modules, circuits, and steps can be achieved through wired electronic signals, wireless electromagnetic signals or optical signals in a direct or an indirect manner. The terms used in the description do not limit the connection type of the present disclosure, and altering the connection type will not deviate from the scope of the present disclosure.

In conclusion, although the present invention has been disclosed with the above embodiments, they are not intended to limit the present invention. Those skilled in the art will appreciate that various modifications and variations can be made without departing from the spirit and scope of the present invention. Therefore, the protection scope of the present invention shall be determined by the following claims.

1. A program execution device comprising:
a program execution module, configured for executing a (z−1)-th round training-program within a (z−1)-th program-training duration, and executing a z-th round training-program within a z-th program-training duration, wherein the (z−1)-th round training-program is an s-th system-selectable program among Y system-selectable programs;
a concentration calculation module, electrically connected to the program execution module, configured for calculating a training-duration concentration level of a (z−1)-th round according to a sensing result corresponding to the (z−1)-th program-training duration and being generated by a sensing device; and
a program selection module, electronically connected to the program execution module and the concentration calculation module, comprising:
a heterogeneous program selection module, electrically connected to the program execution module;
a composite program selection module, electrically connected to the program execution module; and
a selection strategy judgment module, electrically connected to the concentration calculation module, the heterogeneous program selection module, and the composite program selection module, configured for using one of the heterogeneous program selection module and the composite program selection module to select a t-th system-selectable program among the Y system-selectable programs as the z-th round training-program in response to comparison between the training-duration concentration level of the (z−1)-th round and a preset concentration threshold,
wherein z is a positive integer greater than 1, s, t, and Y are positive integers, and s and t are smaller than or equal to Y.

2. The program execution device according to claim 1, wherein
when the training-duration concentration level of the (z−1)-th round is lower than or equal to the preset concentration threshold, the heterogeneous program selection module selects the t-th system-selectable program among the Y system-selectable programs as the z-th round training-program; and
when the training-duration concentration level of the (z−1)-th round is higher than the preset concentration threshold, the composite program selection module selects the t-th system-selectable program among the Y system-selectable programs as the z-th round training-program.

3. The program execution device according to claim 1, wherein the program execution module executes a u-th system-selectable program for a first program-training duration, and the program execution module randomly selects u among positive integers smaller than or equal to Y prior to the first program-training duration.

4. The program execution device according to claim 1, wherein
the Y system-selectable programs correspond to Y sets of program training-aspect contributing effects, respectively, wherein the program execution device further comprises:
a program similarity calculation module, electrically connected to the heterogeneous program selection module and the composite program selection module, configured for calculating Y sets of program similarity degrees between an s-th set of program training-aspect contributing effect corresponding to the s-th system-selectable program and the Y sets of program training-aspect contributing effects.

5. The program execution device according to claim 4, wherein
when the heterogeneous program selection module selects the t-th system-selectable program among the Y system-selectable programs as the z-th round training-program, s is not equal to t.

6. The program execution device according to claim 4, wherein
the t-th system-selectable program corresponds to a t-th set of program similarity degree among the Y sets of program similarity degrees, and
the t-th set of program similarity degree, among the Y sets of program similarity degrees, is lower than other (Y−1) sets of program similarity degrees corresponding to other (Y−1) system-selectable programs.

7. The program execution device according to claim 4, wherein
a y-th set of program training-aspect contributing effect among the Y sets of program training-aspect contributing effects represents training effects regarding X training aspects provided by a y-th system-selectable program among the Y system-selectable programs,
wherein y is a positive integer, and y is smaller than or equal to Y.

8. The program execution device according to claim 4, further comprising:
a program completion calculation module, electrically connected to the program execution module and the composite program selection module, configured for calculating a training-program completion rate of the (z−1)-th round according to a scoring result of the (z−1)-th round generated by the program execution module in response to an operation performed by a user within the (z−1)-th program-training duration, wherein
when the composite program selection module selects the t-th system-selectable program among the Y system-selectable programs,
the composite program selection module selects the t-th system-selectable program according to the training-duration concentration level of the (z−1)-th round and the training-program completion rate of the (z−1)-th round.

9. The program execution device according to claim 8, wherein
the concentration calculation module calculates a training-duration concentration level of a z-th round according to a sensing result of the z-th round corresponding to the z-th program-training duration and generated by the sensing device,
the program execution module generates an execution result of the z-th round in response to an operation performed by the user within the z-th program-training duration, and
the program completion calculation module calculates a training-program completion rate of the z-th round according to the execution result of the z-th round, wherein the program execution device further comprises:
a training result calculation module, comprising:
a participation factor calculation module, electrically connected to the concentration calculation module and the program completion calculation module, configured for calculating a user participation factor of the z-th round according to the training-duration concentration level of the z-th round and the training-program completion rate of the z-th round;
an actual training-volume calculation module, electrically connected to the participation factor calculation module and the composite program selection module, configured for calculating a user actual training-volume of the z-th round according to a t-th program expected training-volume of the z-th round corresponding to the t-th system-selectable program and the user participation factor of the z-th round; and
a remaining training-volume calculation module, electrically connected to the actual training-volume calculation module, configured for calculating a difference between a day's remaining training-volume of the (z−1)-th round and the user actual training-volume of the z-th round as a day's remaining training-volume of the z-th round.

10. The program execution device according to claim 8, wherein
after a first program-training duration, the remaining training-volume calculation module calculates a difference between a target daily training-volume of the user and a user actual training-volume of the first round as a day's remaining training-volume of the first round.

11. The program execution device according to claim 9, wherein the actual training-volume calculation module receives the t-th program expected training-volume of the z-th round from the composite program selection module.

12. The program execution device according to claim 9, wherein the composite program selection module comprises:
a training-volume calculation module, configured for calculating Y program expected training-volumes of the z-th round corresponding to the Y system-selectable programs;
a vector distance calculation module, electrically connected to the training result calculation module and the training-volume calculation module, configured for calculating Y vector space distances of the z-th round between the day's remaining training-volume of the (z−1)-th round and each of the Y program expected training-volumes of the z-th round; and
a vector distance comparison module, electrically connected to the program execution module and the vector distance calculation module, configured for selecting the t-th system-selectable program according to a comparison result of the Y vector space distances of the z-th round.

13. The program execution device according to claim 12, wherein
among the Y vector space distances of the z-th round, a t-th vector space distance of the z-th round corresponding to the t-th system-selectable program is smaller than other (Y−1) vector space distances of the z-th round corresponding to other (Y−1) system-selectable programs.

14. The program execution device according to claim 12, wherein the training-volume calculation module comprises:
an original training-volume calculation module, configured for calculating Y program original training-volumes of the z-th round corresponding to the Y system-selectable programs according to a program-training duration-ratio of the z-th round and the Y sets of program training-aspect contributing effects corresponding to the Y system-selectable programs;
a training-volume adjustment parameter calculation module, configured for using each of Y program training-volume adjustment bases corresponding to the Y system-selectable programs as a base of an exponentiation operation and using each of Y program execution levels corresponding to the Y system-selectable programs as an exponent of the exponentiation operation to perform the exponentiation operation to obtain Y program training-volume adjustment parameters of the z-th round corresponding the Y system-selectable programs; and
an expected training-volume calculation module, configured for multiplying each of the Y program original training-volumes of the z-th round and each of the Y program training-volume adjustment parameters of the z-th round to obtain the Y program expected training-volumes of the z-th round.

15. The program execution device according to claim 14, wherein the composite program selection module further comprises:
a training duration-ratio calculation module, electrically connected to the original training-volume calculation module, configured for calculating a ratio of the z-th program-training duration to a requested daily training duration of the user to obtain the program-training duration-ratio of the z-th round.

16. The program execution device according to claim 15, wherein
the original training-volume calculation module calculates a product of the program-training duration-ratio of the z-th round and each of the Y sets of program training-aspect contributing effects to obtain the Y program original training-volumes of the z-th round.

17. The program execution device according to claim 15, wherein
the Y program execution levels are placed between a system program lowest level and a system program highest level.

18. The program execution device according to claim 17, wherein the composite program selection module further comprises:
a level adjustment module, electrically connected to the program completion calculation module, the training result calculation module, and the training-volume adjustment parameter calculation module,
for selectively adjusting each of the Y program execution levels according to the training-program completion rate of the (z−1)-th round, an upgrade threshold of program completion, a downgrade threshold program completion, and the Y sets of program similarity degrees between the s-th system-selectable program and the Y system-selectable programs posterior to the (z−1)-th program-training duration and prior to the z-th program-training duration,
wherein the upgrade threshold of program completion is higher than the downgrade threshold of program completion.

19. A training system comprising:
a sensing device, configured for sensing within a (z−1)-th program-training duration to generate a sensing result of a (z−1)-th round; and
a program execution device, comprising:
a program execution module, configured for executing a (z−1)-th round training-program within the (z−1)-th program-training duration, and executing a z-th round training-program within a z-th program-training duration, wherein the (z−1)-th round training-program is an s-th system-selectable program among Y system-selectable programs;
a concentration calculation module, electrically connected to the sensing device and the program execution module, configured for calculating a training-duration concentration level of the (z−1)-th round according to the sensing result of the (z−1)-th round; and
a program selection module, electronically connected to the program execution module and the concentration calculation module, comprising:
a heterogeneous program selection module, electrically connected to the program execution module;
a composite program selection module, electrically connected to the program execution module; and
a selection strategy judgment module, electrically connected to the concentration calculation module, the heterogeneous program selection module, and the composite program selection module, configured for using one of the heterogeneous program selection module and the composite program selection module to select a t-th system-selectable program among the Y system-selectable programs as the z-th round training-program in response to comparison between the training-duration concentration level of the (z−1)-th round and a preset concentration threshold,
wherein z is a positive integer greater than 1, s, t, and Y are positive integers, and s and t are smaller than or equal to Y.

20. A program execution method applied to a program execution device, wherein the program execution method comprises steps of:
executing a (z−1)-th round training-program within a (z−1)-th program-training duration, wherein the (z−1)-th round training-program is an s-th system-selectable program among Y system-selectable programs;
calculating a training-duration concentration level of a (z−1)-th round according to a sensing result of the (z−1)-th round corresponding to the (z−1)-th program-training duration;
selecting a t-th system-selectable program among the Y system-selectable programs as a z-th round training-program in response to comparison between the training-duration concentration level of the (z−1)-th round and a preset concentration threshold; and
executing the z-th round training-program within the z-th program-training duration, wherein z is a positive integer greater than 1, s, t, and Y are positive integers, and s and t are smaller than or equal to Y.
